SEQUENCE LISTING (17 sequences)

SEQ ID NO: 1 - protein, 156 aa, Homo sapiens
MEPAAGSSMEPSADWLATAAARGRVEEVRALLEAGALPNAPNSYGRRPIQVMMMGSARVAELLLLHGAEPNCADPATLTRPVHDAAREGFLDTLVVLHRAGARLDVRDAWGRLPVDLAEELGHRDVARYLRAAAGGTRGSNHARIDAAEGPSDIPD

SEQ ID NO: 2 - DNA, 1218 bp, Homo sapiens
cccaacctggggcgacttcaggtgtgccacattcgctaagtgctcggagttaatagcacctcctccgagcactcgctcacggcgtccccttgcctggaaagataccgcggtccctccagaggatttgagggacagggtcggagggggctcttccgccagcaccggaggaagaaagaggaggggctggctggtcaccagagggtggggcggaccgcgtgcgctcggcggctgcggagagggggagagcaggcagcgggcggcggggagcagcatggagccggcggcggggagcagcatggagccttcggctgactggctggccacggccgcggcccggggtcgggtagaggaggtgcgggcgctgctggaggcgggggcgctgcccaacgcaccgaatagttacggtcggaggccgatccaggtcatgatgatgggcagcgcccgagtggcggagctgctgctgctccacggcgcggagcccaactgcgccgaccccgccactctcacccgacccgtgcacgacgctgcccgggagggcttcctggacacgctggtggtgctgcaccgggccggggcgcggctggacgtgcgcgatgcctggggccgtctgcccgtggacctggctgaggagctgggccatcgcgatgtcgcacggtacctgcgcgcggctgcggggggcaccagaggcagtaaccatgcccgcatagatgccgcggaaggtccctcagacatccccgattgaaagaaccagagaggctctgagaaacctcgggaaacttagatcatcagtcaccgaaggtcctacagggccacaactgcccccgccacaacccaccccgctttcgtagttttcatttagaaaatagagcttttaaaaatgtcctgccttttaacgtagatatatgccttcccccactaccgtaaatgtccatttatatcattttttatatattcttataaaaatgtaaaaaagaaaaacaccgcttctgccttttcactgtgttggagttttctggagtgagcactcacgccctaagcgcacattcatgtgggcatttcttgcgagcctcgcagcctccggaagctgtcgacttcatgacaagcattttgtgaactagggaagctcaggggggttactggcttctcttgagtcacactgctagcaaatggcagaaccaaagctcaaataaaaataaaataattttcattcattcactc

SEQ ID NO: 3 - DNA, 36 bp, Artificial Sequence
  Description of Artificial Sequence Synthetic primer
atacgcggatccaccatggagccttcggctgactgg

SEQ ID NO: 4 - DNA, 68 bp, Artificial Sequence
  Description of Artificial Sequence Synthetic primer
aaatttaaagcggccgctcagctagcgtaatctggtacgtcgtatgggtaatcggggatgtctgaggg

SEQ ID NO: 5 - DNA, 19 bp, Artificial Sequence
  Description of Artificial Sequence Synthetic oligonucleotide
caacgcaccgaatagttac

SEQ ID NO: 6 - DNA, 19 bp, Artificial Sequence
  Description of Artificial Sequence Synthetic oligonucleotide
cggaaggtccctcagacat

SEQ ID NO: 7 - DNA, 21 bp, Artificial Sequence
  Description of Combined DNA/RNA Molecule Synthetic oligonucleotide
caacgcaccgaauaguuactt

SEQ ID NO: 8 - DNA, 21 bp, Artificial Sequence
  Description of Combined DNA/RNA Molecule Synthetic oligonucleotide
guaacuauucggugcguugtt

SEQ ID NO: 9 - DNA, 21 bp, Artificial Sequence
  Description of Combined DNA/RNA Molecule Synthetic oligonucleotide
cggaaggucccucagacautt

SEQ ID NO: 10 - DNA, 21 bp, Artificial Sequence
  Description of Combined DNA/RNA Molecule Synthetic oligonucleotide
augucugagggaccuuccgtt

SEQ ID NO: 11 - DNA, 21 bp, Artificial Sequence
  Description of Combined DNA/RNA Molecule Synthetic oligonucleotide
caacgcaccuaauaguuactt

SEQ ID NO: 12 - DNA, 21 bp, Artificial Sequence
  Description of Combined DNA/RNA Molecule Synthetic oligonucleotide
guaacuauuaggugcguugtt

SEQ ID NO: 13 - DNA, 21 bp, Artificial Sequence
  Description of Combined DNA/RNA Molecule Synthetic oligonucleotide
ggcuacguccaggagcgcacc

SEQ ID NO: 14 - DNA, 21 bp, Artificial Sequence
  Description of Combined DNA/RNA Molecule Synthetic oligonucleotide
ugcgcuccuggacguagccuu

SEQ ID NO: 15 - DNA, 21 bp, Artificial Sequence
  Description of Combined DNA/RNA Molecule Synthetic oligonucleotide
gcugcaccugacgcccuuctt

SEQ ID NO: 16 - DNA, 22 bp, Artificial Sequence
  Description of Combined DNA/RNA Molecule Synthetic oligonucleotide
gaaagggcgucaggugcagctt

SEQ ID NO: 17 - protein, 155 aa, Homo sapiens
MEPAAGSSMEPSADWLATAAARGRVEEVRALLEAGALPNAPNSYGRRPIQVMMMGSARVAELLLLHAEPNCADPATLTRPVHDAAREGFLDTLVVLHRAGARLDVRDAWGRLPVDLAEELGHRDVARYLRAAAGGTRGSNHARIDAAEGPSDIPD

(19) United States
(12) Patent Application Publication
(10) Pub. No.: US 2006/0188507 A1
(43) Pub. Date: Aug. 24, 2006

(54) Methods for identifying chemotherapeutic resistance in non-hematopoietic tumors

(76) Inventors: Elias Georges, Laval (CA); Panagiotis Prinos, Montreal (CA)

Correspondence Address: WILMER CUTLER PICKERING HALE AND DORR LLP, 60 STATE STREET, BOSTON, MA, 02109, US

(21) Appl. No.: 11/350,650

(22) Filed: Feb. 9, 2006

Related U.S. Application Data
(60) Provisional application No. 60/652,016, filed on Feb. 11, 2005.

(51) Int. Cl. C12Q 1/68; A61K 39/395; A61K 48/00; A61K 31/704

(52) U.S. Cl. 424/155.1; 435/6; 514/34; 514/44

(57) ABSTRACT

Disclosed are methods for detecting adriamycin resistance in a test neoplastic cell from a non-hematological cancer. The methods include detecting a level of p16 expression in the test neoplastic cell of a given origin or cell type, and comparing the level of p16 expression detected in the test neoplastic cell to the level of p16 expression in a nonresistant neoplastic cell of the same origin or cell type, wherein the test neoplastic cell is adriamycin resistant if the level of p16 expression is greater than the level of p16 expression in the nonresistant neoplastic cell of the same origin or cell type. Also disclosed are therapeutic compositions comprising an agent that inhibits p16 and a pharmaceutically acceptable carrier.

This Application claims the benefit of priority to U.S. Provisional Application No. 60/652,016, filed Feb. 11, 2005, the specification of which is incorporated by reference in its entirety.

### FIELD OF THE INVENTION

The invention relates to the treatment of cancer. In particular, this invention is related to the detection, diagnosis, and treatment of cancer and/or multi-drug resistant cancer.

### BACKGROUND OF THE INVENTION

Millions of people are currently afflicted with cancer, which is one of the leading causes of death among Americans. Cancer is caused by the abnormal growth and proliferation of cells in the body. While normal body cells grow, divide, and die in an orderly fashion, neoplastic (i.e., cancerous) cells grow and divide in a disorderly fashion. Although cancer is frequently fatal, rapid and effective treatment of the disease results in a better prognosis for recovery.

At present, cancer patients are often treated with chemotherapeutic agents. One class of chemotherapeutic agents, the anthracylines, such as adriamycin (ADR; also called doxorubicin) and epirubicin (Ellence®), is widely used in cancer therapy in the treatment of leukemias and solid tumors (Rathore and Elfenbein, Med. Health R. L 8:240-242, 2003; Herzog et al., Gynecol. Oncol. 3 Pt 2:S45-50, 2003; O'Shaughnessy J., Oncologist 8 Suppl. 2:1-2, 2003; Schally and Nagy A, Life Sci. 72: 2305-2320, 2003). The cytotoxicity of adriamycin is thought to be due to several mechanisms including DNA intercalation and cleavage, and generation of superoxides and toxic radicals that damage DNA (Quiles et al., Toxicology 180:79-95, 2002; Tewey et al., Science 226: 466468, 1984; Nakazawa et al., Biochem. Pharmacol. 34: 481-90, 1985; Tritton, T. R., Pharmacol. Ther. 49: 293-309, 1991).

However, the rise of drug resistance in tumor cells limits the successful outcome of chemotherapeutic treatment of cancer patients. Multidrug resistance (MDR) occurs through a number of mechanisms (reviewed in Baker and El-Osta, Exp. Cell. Res. 290:177-194, 2003; Hirose et al., J. Med. Invest. 50:126-135 2003; Efferth et al., Blood Cells Mol. Dis. 28:47-56, 2002; Mossink et al., Oncogene. 22: 7458-7467, 2003). The mechanisms proposed for adriamycin-specific drug resistance and MDR obtained with adriamycin include: (a) overexpression of ABC membrane transporters (e.g., P-170 glycoprotein (P-gpl or ABCC1)), the multidrug resistance protein (MRP or ABCB1), and/or breast cancer resistance protein (BCRP or ABCG2) causing enhanced drug efflux (Warmann et al., Anticancer Res. 23(6C): 46074611, 2003; Marzolini et al., Clin. Pharmacol. Ther. 75:13-33, 2004; Ambudkar et al., Oncogene 22: 7468-7485, 2003), and (b) increased glutathionine concentration and overexpression of glutathione transferase (L'Ecuyer et al., Am. J. Physiol. Heart Circ. Physiol. 286(6): H2057-64, 2004), and apoptosis related proteins p53 and BCL2 (Taniguchi, T. et al., Leukemia 13: 1760-1769, 1999, and Yeh, P. Y. et al., Oncogene 29: 3580-3588, 2004). However, none of these proposed mechanisms has yielded a successful therapy for reversing adriamycin and/or multidrug resistance in tumor cells.

Thus, there is a need for developing methods and compositions for combating adriamycin and/or multidrug resistance in cancer cells.

Interestingly, the p16^{INK4a} (p16) tumor suppressor (also known as MTS1) is one of the most commonly affected gene in cancerous tumors. P16 belongs to the INK4 family of cdk inhibitor proteins, which also includes p14^{INK4a}, p15^{INK4b} (p15), p18^{INK4c} (p18), and p19^{INK4d} (p19) (Ortega et al., Biochim Biophys Acta. 1602:73-87, 2002; Drexler, H. G., Leukemia 12:845-859, 1998; Kramer et al., Leukemia 16: 767-775, 2002). The p16^{INK4a} (p16) tumor suppressor protein and other INK4 family members are approximately 50% identical and mediate cell cycle arrest following growth stimulation by cyclin D associated kinases (p16^{INK4a} being an inhibitor of cdk4) (Ortega et al., Biochim Biophys Acta. 1602:73-87, 2002; Drexler H. G., Leukemia 12:845-859, 1998; Kramer et al., Leukemia 16: 767-775, 2002).

p16 is an intracellular, nuclear protein with little or no cell surface expression. Indeed, in healthy cells (as well as adriamycin-sensitive neoplastic cells, as will be shown herein), agents that inhibit p16 (e.g., anti-p16 antibodies, p16 antisense RNA, and p16 siRNA) have little to no p16 protein or p16 mRNA to bind to p16 is the product of the MTS 1 (multi-tumor suppressor 1) gene, the expression of which is often silenced by DNA methylation, point mutation, or deletion in many different types of human tumors and tumor cell lines (see Ortega et al., Biochim Biophys Acta. 1602:73-87, 2002; Obermann et al., J Pathol. 202:252-62, 2004; Ghiorzo et al., Hum. Pathol 35:25-33, 2004; Dalle et al., Blood 99:2620-2623, 2002; Pavey et al., Melanoma Res. 12:539-547, 2002; Tsai et al., J. Oral. Pathol. Med. 30:527-531, 2001; Esposito et al., J. Clin. Pathol. 57:58-63, 2004). p16 expression is inversely correlated with growth rate in normal cells in that it is expressed at highest levels in growth arrested cells, including senescent cells, and at low or undetectable levels in rapidly cycling normal cells (Ortega et al., supra; Bond et al., Exp. Cell. Res. 292:151-156, 2004). While loss of p16 expression was determined to be common in these studies (ranging from 20-50% of tumors), the presence of p16 expression in the nucleus, or overexpression, or aberrant expression was also observed in some tumors and tumors cell lines and was associated variously with metastasis, tumor progression, advanced tumor stage, and in some cases, a good prognosis or a worse prognosis. However, these studies were complicated by several factors: (a) tumor cell lines in general were found to have more deletions than did primary tumors of the same tissue type, suggesting that tissue culture may be selecting for cell types with p16 deletions, and (b) progressive deletions in p16 were found to increase with advancing stage of some tumors and it was not clear whether this was due to drug therapy effects on the tumor that selected for tumor cells containing p16 deletions, or due to tumor progression independent of drug therapy.

It would therefore be useful to further elucidate the role played by p16 and other INK4 family members in tumor progression.

### SUMMARY OF THE INVENTION

The invention is based on the unexpected discovery that p16 overexpression is associated with adriamycin resistance in tumor cell lines. Moreover, silencing of p16 expression in ADR selected tumor cells (i.e., tumor cells that are resistant to adriamycin) with an agent that inhibits p16 expression and/or p16 activity has been found to result in increased sensitivity of drug resistant tumor cells to ADR and other anti-cancer drugs.

Accordingly, in one aspect, the invention features a method of detecting adriamycin resistance in a neoplastic cell from a non-hematological cancer. The method includes measuring a level of p16 expression in the test neoplastic cell of a given origin or cell type, and comparing the level of p16 expression present in the test neoplastic cell to the level of p16 expression in a nonresistant neoplastic cell of the same origin or cell type, where the test neoplastic cell is adriamycin resistant if the level of p16 expression is substantially greater than the level of p16 expression in the nonresistant neoplastic cell of the same origin or cell type.

In one embodiment, the non-hematological cancer is a solid tumor. In some embodiments, the solid tumor is a cancer of a tissue that is, for example, breast, ovary, prostate, brain or lung tissue. In particular embodiments, the level of p16 protein or p16 mRNA is measured. In a particularly useful embodiment, a standardized immunoblot test for determining p16 protein overexpression levels with monoclonal anti-p16 antibodies (SITp16 test) is used to measure the level of p16 protein. In another particularly useful embodiment, the level of p16 mRNA is measured using a standardized RT-PCR test for determining p16 mRNA expression levels (a mRLp16 test).

In a further aspect, the invention provides a method of treating or alleviating an adriamycin resistant non-hematological cancer in a patient comprising administering a therapeutically effective amount of an agent that inhibits p16 to the patient.

In some embodiments, the method comprises administering a therapeutically effective amount of adriamycin to the patient. In certain embodiments, the agent is a p16 siRNA, a p16-specific antibody, or a p16 antisense nucleic acid molecule. In some embodiments, the non-hematological cancer is a solid tumor. In particular embodiments, the solid tumor is a cancer of a tissue that is, for example, breast, ovary, prostate, brain or lung tissue.

In yet another aspect, the invention provides a therapeutic composition comprising an agent that inhibits p16 and a pharmaceutically acceptable carrier. In one embodiment, the composition further comprises adriamycin. In some embodiments, the agent is a p16 siRNA, a p16-specific antibody, or a p16 antisense nucleic acid molecule.

In yet another aspect, the invention provides a method for determining if a cancer in a patient is treatable with adriamycin. The method comprises measuring a level of p16 expression in a test neoplastic cell from the patient, and comparing the level of p16 expression present in the test neoplastic cell from the patient to the level of p16 expression in a nonresistant neoplastic cell of the same origin or cell type, wherein the patient's cancer is not treatable with adriamycin if the level of p16 expression in the test neoplastic cell is greater than the level of p16 expression in the nonresistant neoplastic cell of the same origin or cell type.

In one embodiment, the non-hematological cancer is a solid tumor. In some embodiments, the solid tumor is a cancer of a tissue that is, for example, breast, ovary, prostate, brain or lung tissue. In certain embodiments, the level of p16 protein or the level of p16 mRNA is measured. In a particularly useful embodiment, a standardized immunoblot test for determining p16 protein overexpression levels with monoclonal anti-p16 antibodies (SITp16 test) is used to measure the level of p16 protein. In another particularly useful embodiment, the level of p16 mRNA is measured using a standardized RT-PCR test for determining p16 mRNA expression levels (a mRLp16 test).

In another aspect, the invention provides a method of treating a non-hematological cancer in a cancer patient so as to increase the likelihood of efficacy of a chemotherapeutic agent comprising. The method involves first detecting the presence of adriamycin resistance in a cancer cell from the cancer patient, and then administering to the cancer patient a therapeutically effective amount of an agent that inhibits p16, where adriamycin resistance is determined to be present when the level of p16 expression in the the cancer cell from the cancer patient is greater than the level of p16 expression in a nonresistant cancer cell of the same tissue or cell type. In certain embodiments, the method further includes the step of administering to the cancer patient a therapeutically effective amount of adriamycin.

In some embodiments, the agent that inhibitis p16 is a p16 siRNA, a p16-specific antibody, and/or a p16 antisense nucleic acid molecule. In particular embodiments, the non-hematological cancer to be treated is a solid tumor. In certain particularly useful embodiments, the solid tumor is from the breast, ovary, prostate, brain or lung.

### BRIEF DESCRIPTION OF THE DRAWINGS

FIG. 1A is a schematic diagram showing one non-limiting mechanism for adriamycin-resistance or sensitivity in neoplastic cells.

FIG. 1B is a schematic diagram showing one non-limiting mechanism for adriamycin-resistance or sensitivity in neoplastic cells.

FIG. 1C is a schematic diagram showing one non-limiting mechanism for adriamycin-resistance or sensitivity in neoplastic cells.

FIG. 2 is a schematic diagram of a flow chart showing a number of non-limiting tests that can be employed to assess the p16 protein and/or mRNA (or cDNA) expression level in a cancer cell.

FIG. 3A is a photographic representation of a silver-stained two-dimensional (2D) gel electrophoresis showing the electrophoretic migration of p16 (at tip of arrow) from MCF7 human breast cells.

FIG. 3B is a photographic representation of a silver-stained two-dimensional (2D) gel electrophoresis showing the electrophoretic migration of p16 (at tip of arrow) from MCF7/AR cells (i.e., an adriamycin-resistant version of MCF7 cells).

FIG. 3C is a photographic representation of a silver-stained two-dimensional (2D) gel electrophoresis showing the electrophoretic migration of p16 (at tip of arrow) from MDA-MB-231 human breast cells.

FIG. 3D is a photographic representation of a silver-stained two-dimensional (2D) gel electrophoresis showing the electrophoretic migration of p16 (at tip of arrow) from MDA-MB-231/AR cells (i.e., an adriamycin-resistant version of MDA-MB-231 cells).

FIG. 4 is a graphic representation showing P-glycoprotein mRNA expression in drug sensitive and resistant tumor cells lines where level of mRNA expression in the resistant cell lines is compared to the cell lines' drug sensitive (or parental) cell lines from which they were derived.

FIG. 5A is a representation of a GC-MS spectrogram showing the mass fingerprint obtained for the 2-D gel spot corresponding to p16. The five arrows show the six peptides whose sequence was obtained. (Note that because two of the six peptides overlap in sequence, there are only five, and not six, arrows.).

FIG. 5B shows the amino acid sequences (in single letter code) of the tryptic peptides obtained from the data shown in FIG. 5A aligned with the p16INK4a protein sequence. The peptide sequences (shown here in bold font) were aligned with respect to the entire p16^{INK4a} protein's amino acid sequence identified using the ProFound software search. The balance of this protein's sequence is shown in italicfont.

FIG. 6 is a read-out from the PROFOUND program. These results were used to arrive at the sequence shown in FIG. 5B above

FIG. 7A is a photographic representation of a Western blotting analysis showing the p16 expression in MCF7 cells, MCF7/AR cells, H69 cells, H69/AR cells, PC3 cells, and PC3/Mel cells, where electrophoretically resolved lysates of the cells were immunoblotted with p16-specific antibody.

FIG. 7B is a photographic representation of a Western blotting analysis showing the p16 expression in MDA-MB-231 cells, MDA-MB-231/AR80, MDA-MB-231/AR400, MDA-MB-231/Mito 10 nM, MDA-MB-231/Mito 80 nM, MDA-MB-231/Taxo 2.5 nM, MDA-MB-231 5 nM, Hs578T, BT549, CEM, and SKOV3 cells, where electrophoretically resolved lysates of the cells were immunoblotted with p16-specific antibody.

FIG. 7C is a photographic representation of a Western blotting analysis showing the p16 expression in MCF7, MCF7/AR, MCF7/VLB1, MCF7/VLB10 nM, MCF7/VCR2, MCF7/VCR20 cells, and MCF7/Mito78 nM cells, as well as three different extracts of white blood cells, CEM cells, and SKOV3 cells, and total cell extract from normal mammary gland, where electrophoretically resolved lysates of the cells were immunoblotted with p16-specific antibody.

FIG. 7D is a photographic representation of a Western blotting analysis showing the p16 expression in CEM, CEM/VLB 0.1 μM, CEM/VLB 1 μM, CEM/AR 0.8 μM, CEM/AR 10 μM, MOTL4, MOTL4/VLB 25 nM, MOLT4/AR 250 nM, MOLT4/AR 500 nM cells, K562, and MCF7/AR cells (with MCF7/AR as a positive control), where electrophoretically resolved lysates of the cells were immunoblotted with p16-specific antibody.

FIG. 7E is a photographic representation of a Western blotting analysis showing the p16 expression in MCF7, MCF7/AR, MDA-MB-231, MDA-MB-231/AR, MDA/AR400 nM, H69, H69/AR, and OVCAR cells, as well as in extracts of normal human ovary, prostate, brain and lung tissues), where electrophoretically resolved lysates of the cells were immunoblotted with p16-specific antibody. Note the level of signal from 200 ng/well of purified p16 protein as a marker protein in the left-most lane.

FIG. 8 is a graphic representation showing p16 mRNA expression in the indicated drug sensitive and resistant tumor cell lines where the level of p16 mRNA expression in the resistant cell lines compared to mRNA expression in the drug sensitive (or parental) cell lines from which the resistant cells' were derived.

FIG. 9 is a schematic diagram of a Western blotting analysis of doxorubicin-treated MCF7, MCF7 EC50 Doxo, OVCAR3, and MCR7/AR cells immunoblotted with p16-specific antibody.

FIG. 10A is a representation of a Western blotting analyses showing the effect on expression of p16^{INK4a} protein in HeLa cells following treatment for 24 or 48 hours with the indicated siRNA. p16^{INK4a} protein expression levels were assessed by immunoblotting with p16^{INK4a}-specific monoclonal antibody, α-p16INK4a (Ab4, clone 16P04, JC2; Neomarkers). Expression of control proteins bcl-2 and actin was assessed by immunoblotting with specific anti-bcl-2 (NeoMarkers, Ab-1, Clone 100/05) and anti-actin monoclonal antibodies (pan-actin Ab-5, NeoMarkers, Clone ACTN05) (lower panel).

FIG. 10B is a representation of a Western blotting analyses showing the effect on expression of p16^{INK4a} protein in HeLa cells following treatment for 72 or 96 hours with the indicated siRNA. p16^{INK4a} protein expression levels were assessed by immunoblotting with p16^{INK4a}-specific monoclonal antibody, α-p16INK4a (Ab-4, clone 16P04, JC2; Neomarkers). Expression of control proteins bcl-2 and actin was assessed by immunoblotting with specific anti-bcl-2 (NeoMarkers, Ab-1, Clone 100/05) and anti-actin monoclonal antibodies (pan-actin Ab-5, NeoMarkers, Clone ACTN05) (lower panel).

FIG. 11A is a graphic representations showing the effect of p16 siRNAs treatment on HeLa cell expression of p16^{INK4a} (with two different p16 siRNAs), bcl-2, and GFP proteins after 24 hours post-transfection.

FIG. 11B is a graphic representations showing the effect of p16 siRNAs treatment on HeLa cell expression of p16^{INK4a} (with two different p16 siRNAs), bcl-2, and GFP proteins after 48 hours post-transfection.

FIG. 11C is a graphic representations showing the effect of p16 siRNAs treatment on HeLa cell expression of p16^{INK4a} (with two different p16 siRNAs), bcl-2, and GFP proteins after 72 hours post-transfection.

FIG. 12A is a representation of a Western blotting analysis showing the effects of treatment of MCF7/AR cells with two different p16 siRNAs. Expression of p16^{INK4a} protein is shown, as measured by immunoblotting with p16^{INK4a}-specific monoclonal antibody, α-p16INK4a (Ab-4, Neomarkers clone 16P04, JC2; commercially available from Lab Vision Corp., Fremont, Calif.), 24 and 48 hours post-transfection. Controls included treatment of MCF7/AR cells with bcl-2 and GFP siRNAs, and immunoblotting with specific anti-actin monoclonal antibodies (pan-actin Ab-5, NeoMarkers Clone ACTN05) (see bottom panel).

FIG. 12B is a representation of a Western blotting analysis showing the effects of treatment of MCF7/AR cells with two different p16 siRNAs. Expression of p16^{INK4a} protein is shown, as measured by immunoblotting with p16^{INK4a}-specific monoclonal antibody, α-p16INK4a (Ab-4, Neomarkers clone 16P04, JC2; commercially available from Lab Vision Corp., Fremont, Calif.), 72 and 96 hours post-transfection. Controls included treatment of MCF7/AR cells with bcl-2 and GFP siRNAs, and immunoblotting with specific anti-actin monoclonal antibodies (pan-actin Ab-5, NeoMarkers Clone ACTN05) (see bottom panel).

FIG. 13A is a graphic representation showing the effect of p16 protein expression in MCF7/AR cells transfected 24 hours previously with p16^{INK4a} siRNA (with two different p16 siRNAs), bcl-2 siRNA, and GFP siRNA.

FIG. 13B is a graphic representation showing the effect of p16 protein expression in MCF7/AR cells transfected 48 hours previously with p16^{INK4a} siRNA (with two different p16 siRNAs), bcl-2 siRNA, and GFP siRNA.

FIG. 13C is a graphic representation showing the effect of p16 protein expression in MCF7/AR cells transfected 72 hours previously with p16^{INK4a} siRNA (with two different p16 siRNAs), bcl-2 siRNA, and GFP siRNA.

FIG. 13D is a graphic representation showing the effect of p16 protein expression in MCF7/AR cells transfected 96 hours previously with p16^{INK4a} siRNA (with two different p16 siRNAs), bcl-2 siRNA, and GFP siRNA.

FIG. 14A is a graphic representation showing the results of a clonogenic assay assessing the effects on growth of HeLa cells in adriamycin-free media following 4 days post-transfection with the indicated siRNAs (note that the p16 siRNA used is p16 I siRNA).

FIG. 14B is a graphic representation showing the results of a clonogenic assay assessing the effects on growth of MCF7/AR cells following 48 hours post-transfection with the indicated siRNAs (note that the p16 siRNA used is p16 I siRNA).

FIG. 15 is a representation of an agarose gel in which p16 RT-PCT products are resolved according to the p16 mRL test. The levels of p16 mRNA are shown in MCF-7 or MDA-AR cells transfected with either GFP siRNA or p16 siRNA. For comparison purposes, the level of p16 mRNA in untransfected MCF-7 AR, MDA-AR, 2008, and SKOV3 cells was also assessed.

FIG. 16A is a representation of an agarose gel in which p16 RT-PCT products are resolved according to the p16 mRL test from Hela cells transfected 41 hrs previously with gfp siRNA, p16 I siRNA, or bcl-2 siRNA. Equal loading of all lanes was confirmed by the presence of hsp27 mRNA (bottom panel)

FIG. 16B is representations of Western blotting analysis, immunoblotting for p16 protein from Hela cells transfected 41 hrs previously with gfp siRNA, p16 I siRNA, or bcl-2 siRNA. Equal loading of all lanes was confirmed by the presence of hsp27 mRNA (bottom panel).

FIG. 17A is a graphic representation showing the quantitation of the band densities from FIGS. 16A and 16B.

FIG. 17B is a graphic representation showing the quantitation of the band densities from FIG. 16B.

FIG. 18A is a representation of a Western blotting analysis of MDA/AR cells transfected 2 days previously with GFP siRNA, p16 siRNA, or p16 mut siRNA. Equal loading of all lanes was confirmed by blotting for ANX-1 (annexin I).

FIG. 18B is a representation of a Western blotting analysis of MDA/AR cells transfected 2 days or 4 days previously with GFP siRNA, p16 siRNA, or p16 mut siRNA. Equal loading of all lanes was confirmed by blotting for ANX-1 (annexin I).

FIG. 19A is a representation of a Western blotting analysis of MCF7/AR cells transfected 2 days previously with GFP siRNA, p16 siRNA, or p16 mut siRNA. Equal loading of all lanes was confirmed by blotting for ANX-1 (annexin I).

FIG. 19B is a representation of a Western blotting analysis of MCF7/AR cells transfected 2 days or 4 days (FIG. 19B) previously with GFP siRNA, p16 siRNA, or p16 mut siRNA. Equal loading of all lanes was confirmed by blotting for ANX-1.

FIG. 20A is a graphic representation of showing the effects of anti-cancer drugs on HeLa cells transfected with siRNA to GFP (black square), p16 (upward pointed triangle), or Bcl2 (downward pointed triangle). Cells were plated in 96 well plates and 48 hours post-transfection cells were exposed to increasing concentrations of Cisplatium.

FIG. 20B is a graphic representation of showing the effects of anti-cancer drugs on HeLa cells transfected with siRNA to GFP (black square), p16 (upward pointed triangle), or Bcl2 (downward pointed triangle). Cells were plated in 96 well plates and 48 hours post-transfection cells were exposed to increasing concentrations of doxorubicin (or adriamycin).

FIG. 20C is a graphic representation of showing the effects of anti-cancer drugs on HeLa cells transfected with siRNA to GFP (black square), p16 (upward pointed triangle), or Bcl2 (downward pointed triangle). Cells were plated in 96 well plates and 48 hours post-transfection cells were exposed to increasing concentrations of taxol.

FIG. 20D is a graphic representation of showing the effects of anti-cancer drugs on HeLa cells transfected with siRNA to GFP (black square), p16 (upward pointed triangle), or Bcl2 (downward pointed triangle). Cells were plated in 96 well plates and 48 hours post-transfection cells were exposed to increasing concentrations of melphalan.

FIG. 20E is a graphic representation of showing the effects of anti-cancer drugs on HeLa cells transfected with siRNA to GFP (black square), p16 (upward pointed triangle), or Bcl2 (downward pointed triangle). Cells were plated in 96 well plates and 48 hours post-transfection cells were exposed to increasing concentrations of mitoxantrone.

FIG. 21A is a graphic representation showing the effects of anti-cancer drugs on HeLa cells transfected with siRNA to p16 (upward pointed triangle) or Bcl2 (downward pointed triangle). Cells were plated in 96 well plates and 48 hours post-transfection cells were exposed to increasing concentrations of mitomycin C.

FIG. 21B is a graphic representation showing the effects of anti-cancer drugs on HeLa cells transfected with siRNA to p16 (upward pointed triangle) or Bcl2 (downward pointed triangle). Cells were plated in 96 well plates and 48 hours post-transfection cells were exposed to increasing concentrations of thio tepa

FIG. 21C is a graphic representation showing the effects of anti-cancer drugs on HeLa cells transfected with siRNA to p16 (upward pointed triangle) or Bcl2 (downward pointed triangle). Cells were plated in 96 well plates and 48 hours post-transfection cells were exposed to increasing concentrations of chlorambucil.

FIG. 22A is a graphic representation showing the effects of anti-cancer drugs on MCF7/AR cells transfected with siRNA to GFP (black square) or p16 (upward pointed triangle). Cells were plated in 96 well plates and 48 hours post-transfection cells were exposed to increasing concentrations of cisplatinum.

FIG. 22B is a graphic representation showing the effects of anti-cancer drugs on MCF7/AR cells transfected with siRNA to GFP (black square) or p16 (upward pointed triangle). Cells were plated in 96 well plates and 48 hours post-transfection cells were exposed to increasing concentrations of taxol.

FIG. 22C is a graphic representation showing the effects of anti-cancer drugs on MCF7/AR cells transfected with siRNA to GFP (black square) or p16 (upward pointed triangle). Cells were plated in 96 well plates and 48 hours post-transfection cells were exposed to increasing concentrations of vinblastine.

FIG. 22D is a graphic representation showing the effects of anti-cancer drugs on MCF7/AR cells transfected with siRNA to GFP (black square) or p16 (upward pointed triangle). Cells were plated in 96 well plates and 48 hours post-transfection cells were exposed to increasing concentrations of chlorambucil.

FIG. 22E is a graphic representation showing the effects of anti-cancer drugs on MCF7/AR cells transfected with siRNA to GFP (black square) or p16 (upward pointed triangle). Cells were plated in 96 well plates and 48 hours post-transfection cells were exposed to increasing concentrations of vincristine.

FIG. 22F is a graphic representation showing the effects of anti-cancer drugs on MCF7/AR cells transfected with siRNA to GFP (black square) or p16 (upward pointed triangle). Cells were plated in 96 well plates and 48 hours post-transfection cells were exposed to increasing concentrations of thio-tepa.

FIG. 23A is a graphic representation showing the effects of anti-cancer drugs on MCF7/AR cells transfected with p16 I siRNA (upward pointed triangle) and p16 mut siRNA (downward pointed triangle). Cells were plated in 96 well plates and 48 hours post-transfection cells were exposed to increasing concentrations of vincristine.

FIG. 23B is a graphic representation showing the effects of anti-cancer drugs on MCF7/AR cells transfected with p16 I siRNA (upward pointed triangle) and p16 mut siRNA (downward pointed triangle). Cells were plated in 96 well plates and 48 hours post-transfection cells were exposed to increasing concentrations of taxol.

FIG. 23C is a graphic representation showing the effects of anti-cancer drugs on MCF7/AR cells transfected with p16 I siRNA (upward pointed triangle) and p16 mut siRNA (downward pointed triangle). Cells were plated in 96 well plates and 48 hours post-transfection cells were exposed to increasing concentrations of cisplatinum.

FIG. 23D is a graphic representation showing the effects of anti-cancer drugs on MCF7/AR cells transfected with p16 I siRNA (upward pointed triangle) and p16 mut siRNA (downward pointed triangle). Cells were plated in 96 well plates and 48 hours post-transfection cells were exposed to increasing concentrations of adriamycin (doxorubicin).

FIG. 24A is a graphic representation showing the effects of anti-cancer drugs on MDA/AR cells transfected with p16 I siRNA (upward pointed triangle) and p16 mut siRNA (downward pointed triangle). Cells were plated in 96 well plates and 48 hours post-transfection cells were exposed to increasing concentrations of vincristine.

FIG. 24B is a graphic representation showing the effects of anti-cancer drugs on MDA/AR cells transfected with p16 I siRNA (upward pointed triangle) and p16 mut siRNA (downward pointed triangle). Cells were plated in 96 well plates and 48 hours post-transfection cells were exposed to increasing concentrations of taxol.

FIG. 24C is a graphic representation showing the effects of anti-cancer drugs on MDA/AR cells transfected with p16 I siRNA (upward pointed triangle) and p16 mut siRNA (downward pointed triangle). Cells were plated in 96 well plates and 48 hours post-transfection cells were exposed to increasing concentrations of cisplatinum.

FIG. 24D is a graphic representation showing the effects of anti-cancer drugs on MDA/AR cells transfected with p16 I siRNA (upward pointed triangle) and p16 mut siRNA (downward pointed triangle). Cells were plated in 96 well plates and 48 hours post-transfection cells were exposed to increasing concentrations of adriamycin (doxorubicin).

FIG. 25A is a graphic representation showing the effects of anti-cancer drugs on MDA/AR cells transfected with p16 I siRNA (upward pointed triangle) and p16 mut siRNA (downward pointed triangle). Cells were plated in 96 well plates and 48 hours post-transfection cells were exposed to increasing concentrations of adriamycin (doxorubicin).

FIG. 25B is a graphic representation showing the effects of anti-cancer drugs on MDA/AR cells transfected with p16 I siRNA (upward pointed triangle) and p16 mut siRNA (downward pointed triangle). Cells were plated in 96 well plates and 48 hours post-transfection cells were exposed to increasing concentrations of cisplatinum.

FIG. 25C is a graphic representation showing the effects of anti-cancer drugs on MDA/AR cells transfected with p16 I siRNA (upward pointed triangle) and p16 mut siRNA (downward pointed triangle). Cells were plated in 96 well plates and 48 hours post-transfection cells were exposed to increasing concentrations of taxol.

FIG. 25D is a graphic representation showing the effects of anti-cancer drugs on MDA/AR cells transfected with p16 I siRNA (upward pointed triangle) and p16 mut siRNA (downward pointed triangle). Cells were plated in 96 well plates and 48 hours post-transfection cells were exposed to increasing concentrations of vincristine.

FIG. 26A is a graphic representation showing the effects of anti-cancer drugs on MDA/AR cells transfected with p16 I siRNA (upward pointed triangle) and p16 mut siRNA (downward pointed triangle). Cells were plated in 96 well plates and 48 hours post-transfection cells were exposed to increasing concentrations of thio tepa.

FIG. 26B is a graphic representation showing the effects of anti-cancer drugs on MDA/AR cells transfected with p16 I siRNA (upward pointed triangle) and p16 mut siRNA (downward pointed triangle). Cells were plated in 96 well plates and 48 hours post-transfection cells were exposed to increasing concentrations of cisplatinum.

FIG. 26C is a graphic representation showing the effects of anti-cancer drugs on MDA/AR cells transfected with p16 I siRNA (upward pointed triangle) and p16 mut siRNA (downward pointed triangle). Cells were plated in 96 well plates and 48 hours post-transfection cells were exposed to increasing concentrations of taxol.

FIG. 26D is a graphic representation showing the effects of anti-cancer drugs on MDA/AR cells transfected with p16 I siRNA (upward pointed triangle) and p16 mut siRNA (downward pointed triangle). Cells were plated in 96 well plates and 48 hours post-transfection cells were exposed to increasing concentrations of adriamycin (doxorubicin).

FIG. 26E is a graphic representation showing the effects of anti-cancer drugs on MDA/AR cells transfected with p16 I siRNA (upward pointed triangle) and p16 mut siRNA (downward pointed triangle). Cells were plated in 96 well plates and 48 hours post-transfection cells were exposed to increasing concentrations of vincristine.

### DETAILED DESCRIPTION

All of the patents and printed publications cited herein reflect the knowledge in the art and are hereby incorporated by reference in their entirety to the same extent as if each was specifically stated to be incorporated by reference. Any inconsistency between these patents and printed publications and the present disclosure shall be resolved in favor of the present disclosure.

The invention provides methods for determining if a neoplastic cell is responsive (i.e., sensitive) to a chemotherapeutic agent, such as adriamycin. The invention also provides methods for making a chemotherapeutic-resistant neoplastic cell more sensitive to that chemotherapeutic by administering to the cell an agent that inhibits p16 expression and/or p16 activity.

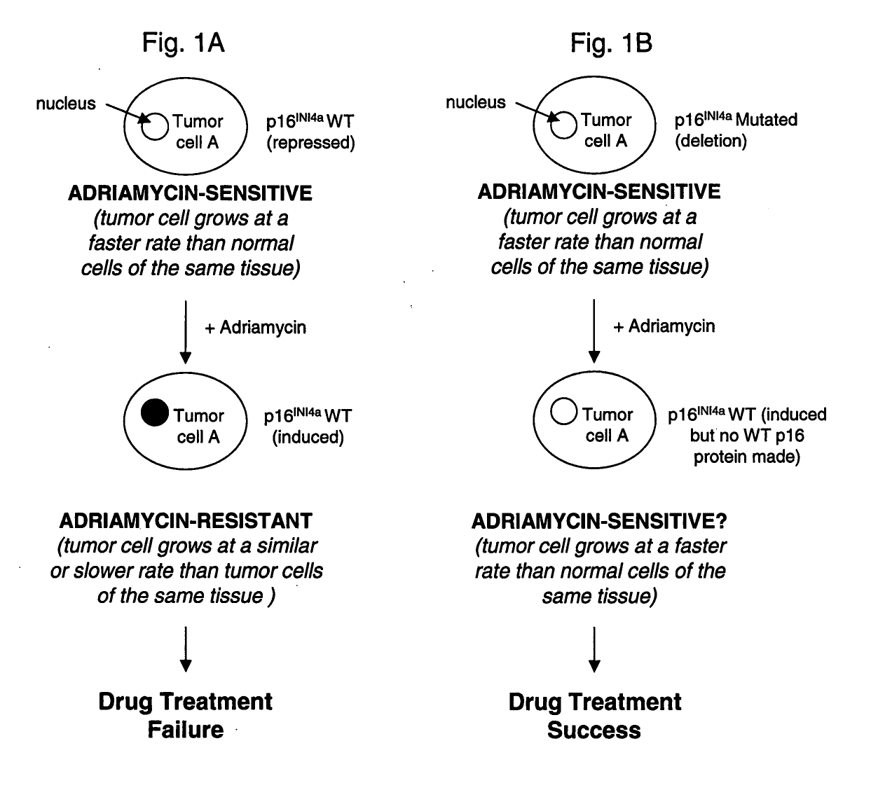

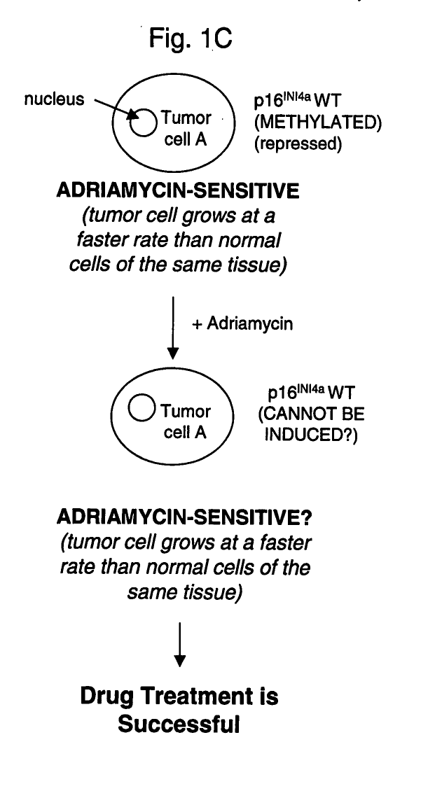

The invention stems from the discovery that tumor cells that are selected with adriamycin (and so are resistant to adriamycin), but not vinblastine vincristine or mitoxantrone, show overexpression of p16. FIGS. 1A-1C show non-limiting examples of a non-limiting mechanism by which adriamycin-resistance may arise. In FIG. 1A, the p16 gene, which is naturally expressed at very low quantities, if at all, is induced (i.e., is expressed) when the tumor cell is treated with adriamycin and becomes resistant to adriamycin. While not wishing to be held to a single mechanism to explain the underlying mechanism by which the invention functions, we believe that, because of this higher level of p16 expression, the tumor cell tends to grow more slowly in general and becomes adriamycin-resistant, such that the it grows at a similar or slower rate than adriamycin-sensitive tumor cells of the same tissue type when adriamycin is not present.

In FIG. 1B, the p16 gene is mutated such that it can never be expressed. Although the gene would normally have been induced when the tumor cell is treated with, and becomes resistant to, adriamycin, because the gene is actually mutated, no p16 protein can be generated from the gene. With no p16 protein, the cell remains sensitive to adriamycin and grows at a faster rate than normal cells of the same tissue. This shows that p16 protein function (and not just elevation of p16 mRNA levels) is required for the acquisition of adriamycin resistance.

FIG. 1C is a schematic diagram showing a cell in which the p16 gene is silenced (in this non-limiting example, by methylation). Upon exposure to adriamycin, depending upon how the gene is silenced, it may or may not be induced. Accordingly, if the gene is not induced, the cell becomes adriamycin-sensitive; however, if the gene is induced (i.e., if p16 protein is made), then the cell becomes adriamycin-resistant.

Based on these findings, the invention allows for the identification of cancer cells that are resistant to a chemotherapeutic agent, such as adriamycin. In addition, the invention provides methods for determining if a cancer in a patient will be treatable with a chemotherapeutic agent, such as adriamycin. Further, the invention provides methods for rendering a neoplastic cell that is resistant to a chemotherapeutic agent responsive or sensitive to that chemotherapeutic agent.

Accordingly, an aspect of the invention features a method of detecting resistance to a chemotherapeutic agent in a neoplastic cell. In some embodiments, the neoplastic cell is derived from a non-hematological tumor. The method includes measuring a level of p16 expression in the test neoplastic cell of a given origin or cell type, and comparing the level of p16 expression present in the test neoplastic cell to the level of p16 expression in a nonresistant neoplastic cell of the same origin or cell type, wherein the test neoplastic cell is resistant to the chemotherapeutic agent if the level of p16 expression is substantially greater than the level of p16 expression in the nonresistant neoplastic cell of the same origin or cell type.

As used herein, the term “chemotherapeutic agent” or simply “chemotherapeutic” means a chemical used to treat or relieve the symptoms of a patient with cancer. One non-limiting example of a chemotherapeutic is adriamycin, which is also called doxorubicin. Other non-limiting chemotherapeutic agents of the invention include cisplatinum, taxol, vinblastin, chlorambucil, vincristin, thio tepa, melphalan, mytomycin C, and mitoxantrone.

As used herein, by “origin or cell type” is meant the originating organ or tissue of the neoplastic cell, or type of cell from which the neoplastic cell is derived. For example, the neoplastic cell may be derived from a liver cell, a skin cell, a breast cell, or a prostate cell. By the “same origin or cell type” is meant that two cells are derived from the same type of organ (e.g., liver cells), or have the same path of development (e.g., lymphocytes, neurons, epithelial cells).

By “resistant” or “resistance” is meant that a neoplastic cell is not sensitive to a clinically approved dosage of the indicated chemotherapeutic agent. In some embodiments, the resistant cell does not show a retardation in cell growth and/or cell division, an abrogation in cell growth and/or cell division, or cell death upon exposure to the same amount of the chemotherapeutic agent that would cause retardation in cell growth and/or cell division, abrogation in cell growth and/or cell division, or cell death of a sensitive or responsive cell of the same origin or cell type upon exposure. In some embodiments, 5-20 fold more of the chemotherapeutic agent is required to see a response, such as growth retardation, by a resistant cell than by sensitive cell of the same origin or cell type.

As used herein, by “sensitive” is meant that a neoplastic cell responds (e.g., by a retardation in cell growth and/or cell division, an abrogation in cell growth and/or cell division, or cell death) to a clinically approved dosage of the indicated chemotherapeutic, such as adriamycin.

In one embodiment of the invention, the chemotherapeutic agent is adriamycin. Thus, in one particular embodiment, a neoplastic cell is defined as being adriamycin resistant if it is more resistant to adriamycin (i.e., does not show a retardation in cell growth and/or cell division, an abrogation in cell growth and/or cell division, or cell death) than an adriamycin sensitive neoplastic cell of the same origin or cell type. In one example, an adriamycin sensitive neoplastic cell may respond to adriamycin at an EC50 (i.e., molar concentration of an agent, which produces 50% of the maximum possible response for that agent) of 1.5 μM or less. In another example, an adriamycin sensitive neoplastic cell responds to adriamycin at an EC50 of 1.0 μM or less. In another example, an adriamycin sensitive neoplastic cell responds to adriamycin at an EC50 of 0.5 μM or less. In some embodiments, adriamycin sensitivity and resistance is determined according to the SART test described below in the Examples. In some embodiments, an adriamycin sensitive cell has a SART score of less than 50 nM. In some embodiments, an adriamycin sensitive cell has a SART score of less than 20 nM. In some embodiments, an adriamycin-resistant cell is sensitive to 5-20 fold more adriamycin than an adriamycin-sensitive cell of the same origin or cell type.

As used herein, by “hematological tumor” is meant a tumor that has arisen from a blood cell. In accordance with the invention, the hematological tumor may be non-solid, such as a leukemia, or a solid tumor, such as a lymphoma.

As used herein, by “non-hematological tumor” is meant a tumor that is not derived from a blood cell. Non-limiting examples of non-hematological tumors of the invention include breast cancer, prostate cancer, brain cancer, cancers of the digestive tracts (e.g., colon cancer or stomach cancer), lung cancer, ovarian cancer, and liver cancer. In a particular embodiment, a neuroglioma (i.e., brain glioma) is not a non-hematological tumor of the invention. In further embodiments, a non-hematological tumor of the invention does not include tumors that are derived from neuronal cells.

In one non-limiting embodiment, a non-hematological tumor of the invention is ovarian carcinoma.

As used herein, the term “neoplastic cell” is used interchangeably with “tumor cell” or “cancer cell” and is used to mean a cell that shows aberrant cell growth, such as increased cell growth. A neoplastic cell may be a hyperplastic cell, a dysplastic cell, a cell that shows a lack of contact inhibition of growth in vitro, a hyperplastic or dysplastic cell that is incapable of metastasis in vivo, or a cell that is capable of metastasis in vivo. In accordance with the invention, a collection of neoplastic cells or tumor cells is called a “tumor”, a “neoplasm”, or a “cancer”. A patient bearing a neoplastic cell in his body is called a cancer patient. A neoplastic cell is “derived” from a cancer patient when that neoplastic cell (or a “parent” of that neoplastic cell) is isolated from the cancer in that cancer patient. One non-limiting method to obtain a neoplastic cell derived from a patient's cancer is to take a biopsy from the patient's cancer, and culture the neoplastic cells from the biopsy in vitro. The cultured cells are thus derived from the patient's cancer.

As described in the Examples below (particularly Example 1 and 2), the levels of ADR-resistance and of p16 protein were determined in a panel of tumor cell lines which were either sensitive or resistant to different extents to ADR or four other cytotoxic drugs, using a standardized test for adriamycin resistance (SART), a standardized immunoblot test for determining p16 protein overexpression levels with monoclonal anti-p16 antibodies (SITp16), and a standardized RT-PCR test for determining p16 mRNA overexpression levels (mRLp16). As described below, solid tumor cell lines from breast, lung, and ovary had SART, SITp16, and mRLp16 scores that corresponded, indicating that the level of ADR resistance could be accurately assessed from the level of p16 protein or mRNA expression in these cell lines. In contrast, SART, SITp16, and mRLp16 scores for hematological tumor cell lines did not correspond, likely due to silencing of the p16 gene by deletion, methylation, or point mutation. Thus, the invention provides a novel mechanism for adriamycin drug-resistance in solid tumor cells that involves overexpression of p16^{INK4A}.

Further, the Examples below describe polymerase chain reactions (PCR) using p16 primers (i.e., the p16 mRL test), in which p16 mRNA was found to be present at very low or negligible levels in adriamycin-sensitive neoplastic cells. In contrast, p16 mRNA was present at 5-20 fold higher levels in adriamycin-resistant or multi-drug resistant (MDR) neoplastic cells. This finding was further confirmed with p16 protein levels. Here, p16 protein was found to be present at 5-20 fold higher levels in adriamycin-resistant or MDR neoplastic cells than in adriamycin-sensitive cells. The overexpression of p16 has been observed previously in a large proportion of both drug-treated and untreated solid tumors, for example, breast and ovarian tumors.

The methods described herein are useful in identifying those patients whose cancers will be responsive to treatment with a chemotherapeutic agent, such as adriamycin. It is, of course, desirable to treat a cancer patient with a chemotherapeutic agent to which the neoplastic cells of the cancer patient will respond. An aspect of the invention provides methods for determining whether the neoplastic cells of the cancer patient will respond to a chemotherapeutic agent, such as adriamycin.

In one non-limiting example, a biopsy is taken from a cancer patient, where the biopsy contains neoplastic cells. The neoplastic cells are then tested, in accordance with the invention, for their level of p16 expression. If the level of p16 expression is low, then the neoplastic cells are likely to be sensitive to a chemotherapeutic agent, such as adriamycin, and the patient can start receiving treatment with adriamycin.

Thus, in a further aspect, the invention provides methods for determining if a cancer in a cancer patient is treatable with a chemotherapeutic agent such as adriamycin. By “treatable” simply means that the cancer patient's symptoms will be treated and/or alleviated by treatment with the chemotherapeutic agent. In other words, a patient is treatable if his neoplastic cells respond to or are sensitive to the chemotherapeutic agent. For example, a cancer is treatable if the neoplastic cells grows and/or metastasize at a rate slower than if the patient was not administered the adriamycin or other chemotherapeutic agent. The method includes detecting a level of p16 expression in the neoplastic cell derived from the patient and comparing the level of p16 expression detected in the test neoplastic cell to the level of p16 expression in a nonresistant neoplastic cell of the same origin or cell type, wherein the patient's symptoms are not likely to be treated and/or alleviated by adriamycin if the level of p16 expression in the patient's neoplastic cells is substantially greater than the level of p16 expression in the nonresistant neoplastic cell of the same origin or cell type.

The level of p16 expression can be assessed at any level, including RNA expression (e.g., mRNA expression), or actual p16 protein expression. The Examples described below (particularly Example 2) provide standardized tests to measure the level of p16 gene overexpression and to correlate with the level of adriamycin resistance tests (SART) in extracts of tumor cell lines and tumor specimens. The level of p16 protein overexpression can also be measured using immunoblotting of 1-dimension gels with total cell protein extracts and anti-p16 antibody (SITp16 test) as well as by immunoblotting of 2-dimension gels of the same extracts. Other non-limiting methods for detecting p16 protein levels are described below and also includes, without limitation, Western blotting analysis, immunoprecipitation using a p16-specific antibody, and measuring p16 protein activity (e.g., its ability to inhibit a cyclin D dependent kinase).

One method of the level of p16 expression is by detecting the level of p16 mRNA. One non-limiting test for measuring the level of p16 mRNA overexpression uses RT-PCR with p16 mRNA specific oligonucleotides (p16 mRL test). Other non-limiting methods for detecting p16 mRNA levels include Northern blotting analysis with a probe specific for p16 mRNA (i.e., the probe recognizes and binds to the spliced p16 gene product).

As described below, these three tests (i.e., the SART, the SITp16 test, and the p16 mRL test) gave scores that corresponding well for solid but not hematological tumor cell lines, indicating that SITp16 and/or p16 mRL are tests that can be used both to monitor the level of ADR drug resistance of a patient's solid tumor, as well as to monitor the effectiveness of p16 siRNA drug resistance reversal therapy.

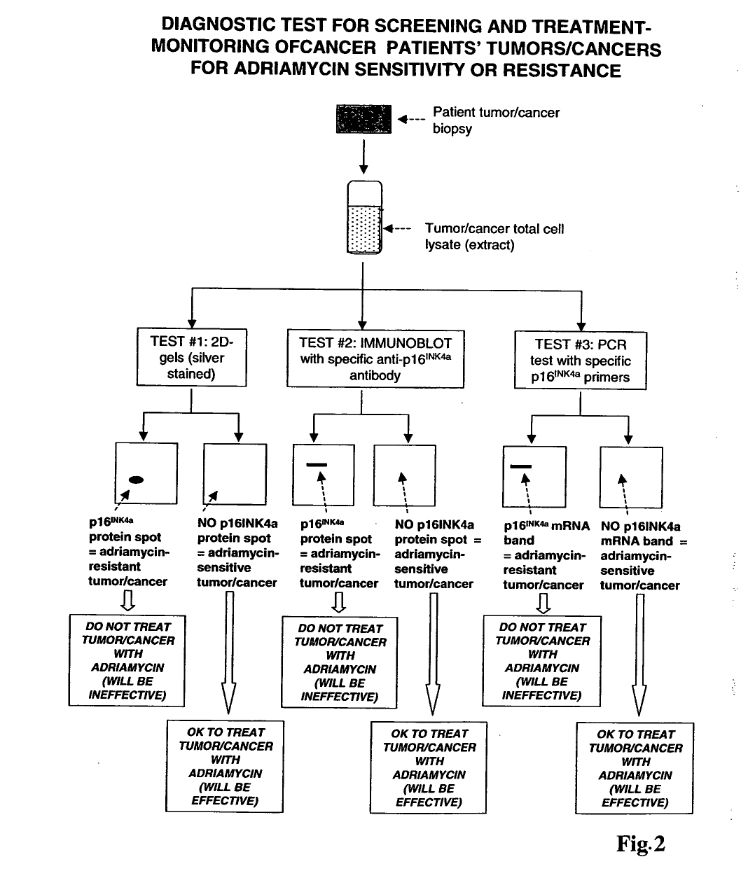

In addition, FIG. 2 provides a schematic flow chart outlining different tests that can be used for assessing if a patient's cancer expresses low levels of p16 and is, thus, adriamycin sensitive. Only those cells that lack expression of the p16 protein and/or mRNA (or cDNA) will be sensitive to adriamycin treatment. As can be seen from FIG. 2, any of three (or all three, or a combination of two) different tests can be employed to assess the level of p16 expression in a patient's neoplastic cells using a lysate prepared from those cells. By “lysate” is meant the internal contents of a cell. A lysate can be produced by puncturing a hole in the cell membrane and extracting the cell's internal contents.

In the first test described in FIG. 2, a two-dimensional (2-D) gel is run on the cell lysate, where the gel containing the resolved lysate proteins are visualized by some matter (e.g., silver staining, Coomassie Blue staining). The appearance of a large spot corresponding to p16 would indicate that the cell from which the lysate was obtained is not adriamycin-sensitive. Thus, there is no need to treat the patient (from whom the cell was obtained) with adriamycin, and other chemotherapeutics (or other therapies) should be attempted.

In the second test described in FIG. 2, the proteins in the lysate are resolved by SDS-PAGE according to well known methods (see, e.g., Ausubel et al., Current Protocols in Molecular Biology, John Wiley & Sons, New York, N.Y. 1988-2004, updated yearly). The gel is then transferred to a nitrocellulose membrane and immunoblotted with an anti-p16 antibody. If the p16 antibody specifically binds to a protein from the cell lysate, this would indicate that the cell from which the lysate was obtained is not adriamycin-sensitive. As used herein, by “specifically binds” is meant that an antibody or other binding agent binds to its target preferentially over other molecules in a mixture of molecules containing the target. In some embodiments, an antibody or binding agent that binds its target with a dissociation constant (Kd) of 10⁻⁶, or 10⁻⁷, or 10⁻⁸, or 10⁻⁹, or 10⁻¹⁰ or lower is an antibody or binding agent that specifically binds to its target.

Another test described in FIG. 2 is a PCR based test in which primers designed to specifically amplify p16 mRNA (or cDNA) are used to amplify DNA isolated from the cell lysate. The PCR product may then be resolved in a standard agarose gel (followed by staining with ethidium bromide or some other agent that allows visualization of nucleic acids) to determine if a PCR product of the expected size is present. There may actually be two different sized p16 PCR products—one from genomic DNA, and one from p16 mRNA or cDNA (e.g., possibly smaller in size since the mRNA and cDNA lack intron sequences). It is the presence of the p16 mRNA or cDNA PCR product that would indicate that the cell from which the lysate was obtained is resistant to adriamycin.

Note that FIG. 2 merely describes three non-limiting tests for p 16 expression, adriamycin-insensitivity which can be performed on lysates prepared from cancer cells. An aspect of the invention, of course, includes other tests for p16 expression, regardless of whether the cell lysate, or entire cell is tested. In one non-limiting example of a test in which the whole cell is used, a cancer cell can be fixed (e.g., with paraformaldehyde), and then permeabilized (e.g., by incubating the fixed cells in a detergent, such as Triton-X). The fixed, permeabilized cell is then intracellularly stained with detectably labeled p16 antibody. The presence of the p16 (as determined by binding of the p16-specific antibody) would indicate that the cell is not adriamycin-sensitive. As used herein, by “detectably labeled” is meant that an antibody (or other agent) is labeled such that it can be visualized by human or mechanical means. For example, a detectably labeled antibody can be covalently coupled to a detectable label, such as a radioactive isotope or a fluorophore (e.g., fluorescein or phycoerythrin). Also included in the definition of a detectably labeled antibody is an antibody that is specifically bound by a secondary antibody, where the secondary antibody is covalently coupled to a detectable label.

Furthermore, older classes of anticancer chemotherapeutic drugs which were abandoned due to the emergence of drug-resistant tumors, may be utilized in combination with specific siRNA that inhibit the emergence of drug-resistant tumors.

Accordingly in another aspect, the invention features a method of detecting chemotherapeutic resistance in a neoplastic cell. In some embodiments, the neoplastic cell may be from a non-hematological tumor. The method includes detecting a level of expression of an INK4 family member in the test neoplastic cell of a given origin or cell type, and comparing the level of expression detected in the test neoplastic cell to the level of expression in a nonresistant neoplastic cell of the same origin or cell type, wherein the test neoplastic cell is resistant to the chemotherapeutic agent if the level of p16 expression is substantially greater than the level of p16 expression in the nonresistant neoplastic cell of the same origin or cell type.

As used herein, the “INK4 family” includes p14^{INK4a} (p14), p15^{INK4b} (p15), p16^{INK4a} (pp16), p19^{INK4d} (p19), and p18^{INK4c} (p18).

Because all of the members of the INK4 family mediate cell cycle arrest following growth stimulation by cyclin D associated kinases, the overexpression of these proteins in a cell is indicative of that cell being insensitive to a chemotherapeutic agent.

The results described in the Examples below also demonstrated that silencing of p16 expression in adriamycin (ADR) selected (i.e., resistant) tumor cells with p16-specific siRNA results in increased sensitivity of drug resistant tumor cells to ADR and other chemotherapeutic agents. For example, although a neoplastic cell may have initially been resistant to adriamycin, once its p16 levels (protein or mRNA) have been reduced, it will become sensitive to adriamycin. In some embodiments, the chemotherapeutic agent to which an adriamycin-resistant cell, upon having its p16 expression level reduced, is more sensitive includes cisplantinum, doxorubicin (i.e., adriamycin), melphalan, mitoxantrone, taxol, vinblastin, chlorambucil, vincristin, and thio tepa. Interestingly, sensitivity to a chemotherapeutic agent can be induced by treating tumor cell lines that are highly resistant to adriamycin and also overexpress P-gpl with p16-siRNA, suggesting that p16 and P-gpl may be related.

Thus, the results in the examples below demonstrate that the p16 mechanism of ADR drug-resistance reversal can be generalized, and taken advantage of to design an expanding arsenal of drug reversal agents to target the expression of other cell cycle control genes that may be overexpressed in drug-resistant cells. For example, siRNAs specific for the INK4 class of cell cycle control genes might be envisioned. In addition, siRNA specific for the retinoblastoma and/or p53 genes are also contemplated, particularly since increased expression of D-type cyclins, or inactivation of INK4 inhibitors, is thought to cause the functional inactivation of Rb, the retinoblastoma gene product which is also a tumor suppressor (Yamasaki, L., Cancer Treat. Res. 115:209-239, 2003). Indeed, p16 is up-regulated in tumors lacking Rb due to a feedback loop in which Rb represses p16 gene expression (Yamasaki, L., supra). Since p16 is frequently wild type (i.e., normal) in tumors with Rb inactivated and vice versa, p16 and Rb may function together in tumor suppression. Tumors with an intact p16/cyclin D1/pRb pathway which had upregulated p16 expression have also been found to have decreased cellular proliferation (Palmqvist et al., supra).

Accordingly, in a further aspect, the invention provides a therapeutic composition comprising an agent that inhibits p16 and a pharmaceutically acceptable carrier. As used herein, terms “agent that inhibits p16” and “p16-inhibitory agent” are used interchangeably and mean an agent capable of decreasing levels of p16 gene expression, mRNA level, protein level or protein activity.

Thus, an aspect of the invention provides a novel class of drug resistance reversal agents, such as a p16 siRNA, that reverses ADR resistance and resistance to other chemotherapeutic agents in ADR-resistant non-hematological solid tumor cell lines (e.g., HeLa and MCF7/AR, MDA-231/AR, and 2008).

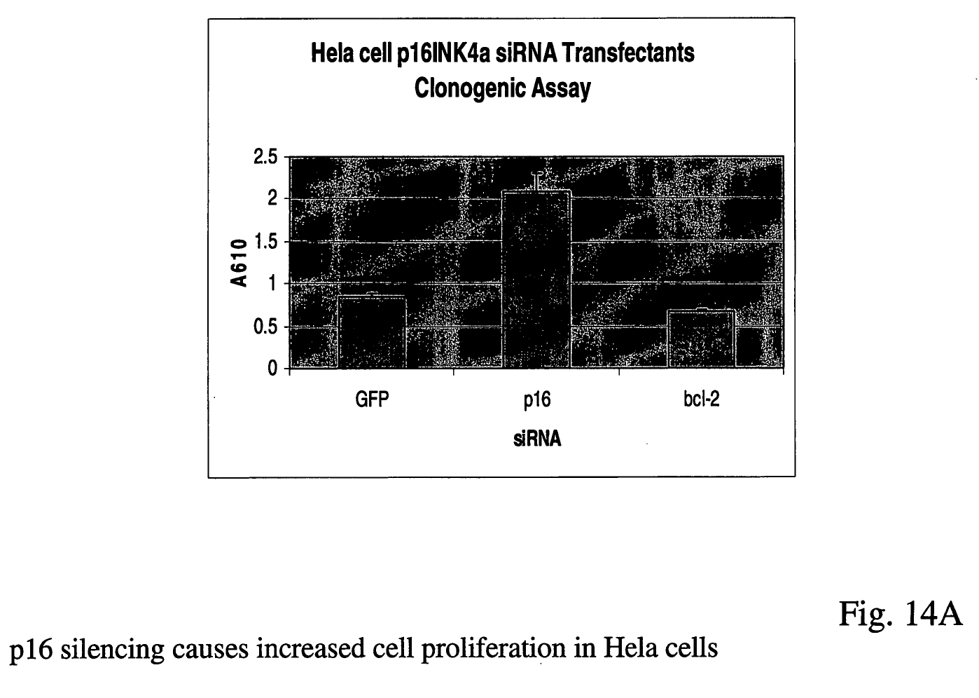

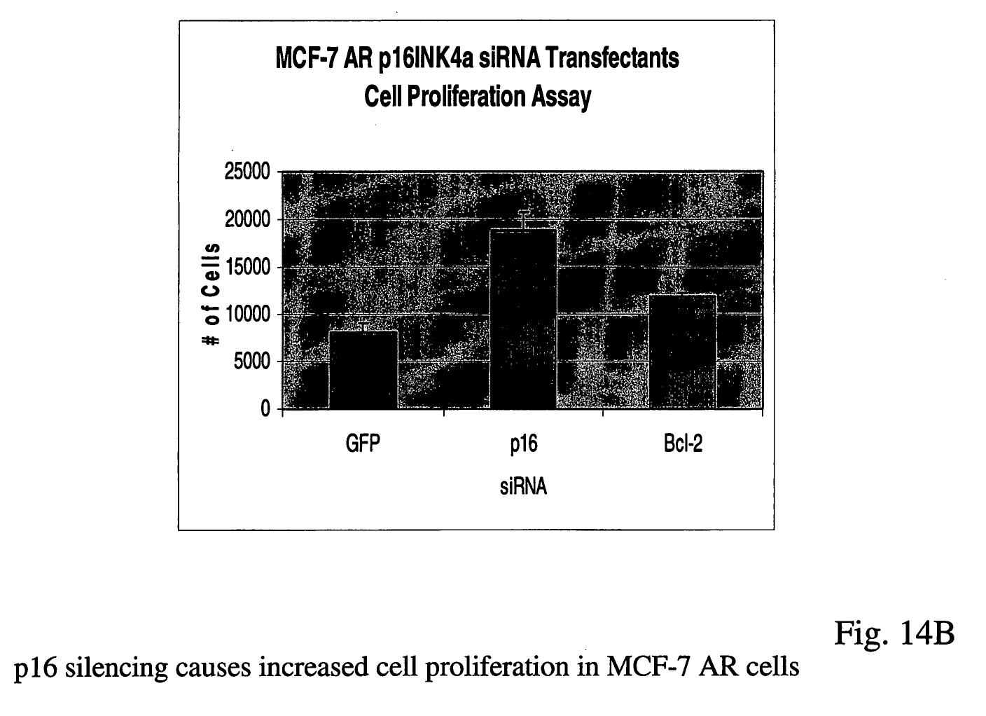

As described in the Examples below, the p16 siRNAs also partially reversed drug resistance to other cancer drug classes. For example, for taxol and cisplatin, a reversal of 1.5 to 2 fold was observed. Thus the p16 gene may act as a positive regulator or “master MDR gene” that coordinately regulates the expression of a number of other MDR genes. The drug-resistance reversal effect of the p16 siRNAs may also be related to their effect on increasing cell proliferation 2-3 fold in transfected cell lines (see, e.g., FIGS. 14A and 14B).

Further, as described in Example 3 below, to determine if p16 is directly involved in drug resistance, two different p16 siRNAs that specifically inhibited p16 protein expression were tested and found to effectively reverse ADR resistance of several tumor cells lines (Hela and 2008) including highly resistant breast cancer cells (MCF7/AR and MDA-231/AR ADR selected cells lines).

Increased sensitivity to other cytotoxic drugs was also enhanced, perhaps due to p16 siRNAs increasing cellular proliferation approximately 2.5 fold. Thus, treatment with p16 siRNA provides a novel drug resistance reversal agent for the treatment of ADR resistant and MDR solid tumors. Moreover, SITp16 and/or mRLp16 tests provide clinically useful information on the level of ADR-drug resistance of a given solid tumor specimen.

Use of an agent that inhibits p16 as a drug resistance modulator/reverser is different than other drug resistance reversal agents previously described (reviewed in Tan et al., Curr Opin Oncol. 12:450-458, 2002) which are designed to push cells down the apoptosis/senescence pathway. While senescent cells are known to have high levels of p16 gene expression (Bond et al., Exp Cell Res. 292:151-156, 2004), the results provided herein suggest that senescent cells may be innately MDR/ADR-resistant.

In some embodiments, a p16-inhibitory agent is a ribozyme or an antisense oligonucleotides.

Ribozymes and antisense oligonucleotides that are targeted to p16 effect p16 inhibition by targeting degradation of the corresponding p16 mRNA and/or by inhibiting protein translation from the messenger RNA. The p16 gene sequence provides useful sequences for the design and synthesis of p16 ribozymes and antisense oligonucleotides. Methods of design and synthesis of ribozymes and antisense oligonucleotides are known in the art. Additional guidance is provided herein.

One issue in designing specific and effective mRNA-targeted ribozymes and antisense oligonucleotides is that of identifying accessible sites of antisense pairing within the target mRNA (which is itself folded into a partially self-paired secondary structure). A combination of computer-aided algorithms for predicting RNA pairing accessibility and molecular screening allow for the creation of specific and effective ribozymes and/or antisense oligonucleotides directed against most mRNA targets. Indeed several approaches have been described to determine the accessibility of a target RNA molecule to antisense or ribozyme inhibitors. One approach uses an in vitro screening assay applying as many antisense oligodeoxynucleotides as possible (see Monia et al., Nature Med. 2:668-675, 1996; and Milner et al., Nature Biotechnol. 15:537-541, 1997). Another utilizes random libraries of antisense oligonucleotides (Ho et al., Nucleic Acids Res. 24:1901-1907, 1996; Birikh et al., RNA 3:429-437, 1997; and Lima et al., J. Biol. Chem. 272:626-638, 1997). The accessible sites can be monitored by RNase H cleavage (see Birikh et al, supra; and Ho et al., Nature Biotechnol. 16:59-63, 1998). RNase H catalyzes the hydrolytic cleavage of the phosphodiester backbone of the RNA strand of a DNA-RNA duplex.

In another approach, a pool of semi-random, chimeric chemically synthesized antisense oligonucleotides is used to identify accessible sites cleaved by RNase H on an in vitro synthesized RNA target. Primer extension analyses are then used to identify these sites in the target molecule (see Lima et al., supra). Other approaches for designing antisense targets in RNA are based upon computer assisted folding models for RNA. Several reports have been published on the use of random ribozyme libraries to screen effective cleavage (see Campbell et al., RNA 1:598-609, 1995; Lieber et al., Mol. Cell Biol. 15: 540-551, 1995; and Vaish et al., Biochem. 36:6459-6501, 1997).

Other in vitro approaches, which utilize random or semi-random libraries of antisense oligonucleotides and RNase H may be more useful than computer simulations (Lima et al., supra). However, use of in vitro synthesized RNA does not predict the accessibility of antisense oligonucleotides in vivo because recent observations suggest that annealing interactions of polynucleotides are influenced by RNA-binding proteins (see Tsuchihashi et al., Science 267:99-102, 1993; Portman et al., EMBO J. 13:213-221, 1994; and Bertrand and Rossi, EMBO J. 13:2904-2912, 1994). U.S. Pat. No. 6,562,570, the contents of which are incorporated herein by reference, provides compositions and methods for determining accessible sites within an mRNA in the presence of a cell extract, which mimics in vivo conditions.

Briefly, this method involves incubation of native or in vitro-synthesized RNAs with defined antisense oligonucleotides, ribozymes or DNAzymes, or with a random or semi-random oligonucleotides, ribozyme or DNAzyme library, under hybridization conditions in a reaction medium which includes a cell extract containing endogenous RNA-binding proteins, or which mimics a cell extract due to presence of one or more RNA-binding proteins. Any antisense oligonucleotides, ribozyme or DNAzyme, which is complementary to an accessible site in the target RNA will hybridize to that site. When defined oligonucleotides or an oligonucleotide library is used, RNase H is present during hybridization or is added after hybridization to cleave the RNA where hybridization has occurred. RNase H can be present when ribozymes or DNAzymes are used, but is not required, since the ribozymes and DNAzymes cleave RNA where hybridization has occurred. In some instances, a random or semi-random oligonucleotide library in cell extracts containing endogenous mRNA, RNA-binding proteins and RNase H is used.

Next, various methods can be used to identify those sites on target RNA to which antisense oligonucleotides, ribozymes or DNAzymes have bound and cleavage has occurred. For example, terminal deoxynucleotidyl transferase-dependent polymerase chain reaction (TDPCR) may be used for this purpose (see Komura and Riggs, Nucleic Acids Res. 26:1807-1811, 1998). A reverse transcription step is used to convert the RNA template to DNA, followed by TDPCR. In this invention, the 3′ termini needed for the TDPCR method is created by reverse transcribing the target RNA of interest with any suitable RNA dependent DNA polymerase (e.g., reverse transcriptase). This is achieved by hybridizing a first oligonucleotide primer (P1) to the RNA in a region which is downstream (i.e., in the 5′ to 3′ direction on the RNA molecule) from the portion of the target RNA molecule which is under study. The polymerase in the presence of dNTPs copies the RNA into DNA from the 3′ end of P1 and terminates copying at the site of cleavage created by either an antisense oligonucleotide/RNase H, a ribozyme or a DNAzyme. The new DNA molecule (referred to as the first strand DNA) serves as first template for the PCR portion of the TDPCR method, which is used to identify the corresponding accessible target sequence present on the RNA.

For example, the TDPCR procedure may then be used, i.e., the reverse-transcribed DNA with guanosine triphosphate (rGTP) is reacted in the presence of terminal deoxynucleotidyl transferase (TdT) to add an (rG)2-4 tail on the 3′ termini of the DNA molecules. Next is ligated a double-stranded oligonucleotide linker having a 3′2-4 overhang on one strand that base-pairs with the (rG)2-4 tail. Then two PCR primers are added. The first is a linker primer (LP) that is complementary to the strand of the TDPCR linker which is ligated to the (rG)2-4 tail (sometimes referred to as the lower strand). The other primer (P2) can be the same as P1, but may be nested with respect to P1, i.e., it is complementary to the target RNA in a region which is at least partially upstream (i.e., in the 3′ to 5′ direction on the RNA molecule) from the region which is bound by P1, but it is downstream of the portion of the target RNA molecule which is under study. That is, the portion of the target RNA molecule, which is under study to determine whether it has accessible binding sites is that portion which is upstream of the region that is complementary to P2. Then PCR is carried out in the known manner in presence of a DNA polymerase and dNTPs to amplify DNA segments defined by primers LP and P2. The amplified product can then be captured by any of various known methods and subsequently sequenced on an automated DNA sequencer, providing precise identification of the cleavage site. Once this identity has been determined, defined sequence antisense DNA or ribozymes can be synthesized for use in vitro or in vivo.

Antisense intervention in the expression of specific genes can be achieved by the use of synthetic antisense oligonucleotide sequences (see, e.g., Lefebvre-d'Hellencourt et al., Eur. Cyokine Netw. 6:7, 1995; Agrawal, S., Trends. Biotechnol. 14: 376, 1996; and Lev-Lehman et al., Antisense Therap., Cohen and Smicek, eds. (Plenum Press, New York 1997)). Briefly, antisense oligonucleotide sequences may be short sequences of DNA, typically 15-30mer but may be as small as 7mer (see Wagner et al., Nature 372: 333, 1994) designed to complement a target mRNA of interest and form an RNA:AS duplex. This duplex formation can prevent processing, splicing, transport or translation of the relevant mRNA. Moreover, certain antisense nucleotide sequences can elicit cellular RNase H activity when hybridized with their target mRNA, resulting in mRNA degradation (see Calabretta et al., Semin. Oncol. 23: 78, 1996). In that case, RNase H will cleave the RNA component of the duplex and can potentially release the antisense olignucleotide to further hybridize with additional molecules of the target RNA. An additional mode of action results from the interaction of antisense olignucleotide with genomic DNA to form a triple helix that may be transcriptionally inactive.

Antisense induced loss-of-function phenotypes related with cellular development have been shown for the glial fibrillary acidic protein (GFAP), for the establishment of tectal plate formation in chick and for the N-myc protein, responsible for the maintenance of cellular heterogeneity in neuroectodermal cultures (ephithelial vs. neuroblastic cells, which differ in their colony forming abilities, tumorigenicity and adherence, see Rosolen et al., Cancer Res. 50: 6316, 1990; and Whitesell et al., Mol. Cell Biol. 11: 1360, 1991). Antisense oligonucleotide inhibition of basic fibroblast growth factor (bFgF), having mitogenic and angiogenic properties, suppressed 80% of growth in glioma cells (see Morrison, J. Biol. Chem. 266: 728, 1991) in a saturable and specific manner.

In as a non-limiting example of, addition to, or substituted for, an antisense sequence as discussed herein above, ribozymes may be utilized for suppression of gene function. This is particularly necessary in cases where antisense therapy is limited by stoichiometric considerations. Ribozymes can then be used that will target the same sequence. Ribozymes are RNA molecules that possess RNA catalytic ability that cleave a specific site in a target RNA. The number of RNA molecules that are cleaved by a ribozyme is greater than the number predicted by a 1:1 stoichiometry (see Hampel and Tritz, Biochem. 28: 4929-4933, 1989; and Uhlenbeck, Nature 328: 596-600, 1987). Therefore, the present invention also allows for the use of the ribozyme sequences targeted to an accessible domain of an Sp1 or Sp3 mRNA species and containing the appropriate catalytic center. The ribozymes are made and delivered as known in the art and discussed further herein. The ribozymes may be used in combination with the antisense sequences.

Ribozymes catalyze the phosphodiester bond cleavage of RNA. Several ribozyme structural families have been identified including Group I introns, RNase P, the hepatitis delta virus ribozyme, hammerhead ribozymes and the hairpin ribozyme originally derived from the negative strand of the tobacco ringspot virus satellite RNA (sTRSV) (see Sullivan, Investig. Dermatolog. (Suppl.) 103: 95S, 1994; and U.S. Pat. No. 5,225,347). The latter two families are derived from viroids and virusoids, in which the ribozyme is believed to separate monomers from oligomers created during rolling circle replication (see Symons, TIBS 14: 445-50, 1989; Symons, Ann. Rev. Biochem. 61: 641-71, 1992). Hammerhead and hairpin ribozyme motifs are most commonly adapted for trans-cleavage of mRNAs for gene therapy. The ribozyme type utilized in the present invention is selected as is known in the art. Hairpin ribozymes are now in clinical trial and are a particularly useful type. In general the ribozyme is from 30-100 nucleotides in length.

Ribozyme molecules designed to catalytically cleave a target mRNA transcript (e.g., a p16 mRNA) can also be used to prevent translation of mRNA (see, e.g., PCT International Pub. WO90/11364; Sarver et al., Science 247:1222-1225, 1990, and U.S. Pat. No. 5,093,246). While ribozymes that cleave mRNA at site specific recognition sequences can be used to destroy particular mRNAs, the use of hammerhead ribozymes is particularly useful. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, Nature 334: 585, 1988).

The ribozymes of the present invention also include RNA endoribonucleases (hereinafter “Cech-type ribozymes”) such as the one which occurs naturally in Tetrahymena thermophila (known as the IVS, or L-19 IVS RNA), and which has been extensively described by Thomas Cech and collaborators (see Zaug et al., Science 224: 574-578, 1984; Zaug and Cech, Science 231: 470-475, 1986; Zaug, et al., Nature 324: 429-433, 1986; PCT Publication No. W088/04300; Been and Cech, Cell 47: 207-216, 1986). The Cech-type ribozymes have an eight base pair active site, which hybridizes to a target RNA sequence where after cleavage of the target RNA takes place. The invention encompasses those Cech-type ribozymes, which target eight base-pair active site sequences. While the invention is not limited to a particular theory of operative mechanism, the use of hammerhead ribozymes in the invention may have an advantage over the use of Sp1/Sp3-directed antisense, as recent reports indicate that hammerhead ribozymes operate by blocking RNA translation and/or specific cleavage of the mRNA target.

As in the antisense approach, the ribozymes can be composed of modified oligonucleotides (e.g., for improved stability or targeting) and are delivered to cells expressing the target mRNA. A useful method of delivery involves using a DNA construct “encoding” the ribozyme under the control of a strong constitutive pol III or pol II promoter, so that transfected cells will produce sufficient quantities of the ribozyme to destroy targeted messages and inhibit translation. Because ribozymes, unlike antisense molecules, are catalytic, a lower intracellular concentration is required for efficiency.

Nuclease resistance, where needed, is provided by any method known in the art that does not substantially interfere with biological activity of the antisense oligodeoxynucleotides or ribozymes as needed for the method of use and delivery (Iyer et al., J. Org. Chem. 55: 4693-4699, 1990; Eckstein, Ann. Rev. Biochem. 54: 367-402, 1985; Spitzer and Eckstein, Nucleic Acids Res. 18: 11691-704, 1988; Woolf et al., Nucleic Acids Res. 18: 1763-1769, 1990; and Shaw et al., Nucleic Acids Res. 18: 11691-1704, 1991). Non-limiting representative modifications that can be made to antisense oligonucleotides or ribozymes in order to enhance nuclease resistance include modifying the phosphorous or oxygen heteroatom in the phosphate backbone, short chain alkyl or cycloalkyl intersugar linkages or short chain heteroatomic or heterocyclic intersugar linkages. These include, e.g., preparing 2′-fluoridated, O-methylated, methyl phosphonates, phosphorothioates, phosphorodithioates and morpholino oligomers. For example, the antisense oligonucleotide or ribozyme may have phosphorothioate bonds linking between four to six 3′-terminus nucleotide bases. Alternatively, phosphorothioate bonds may link all the nucleotide bases. Phosphorothioate antisense oligonucleotides do not normally show significant toxicity at concentrations that are effective and exhibit sufficient pharmacodynamic half-lives in animals (see Agrawal, S., Trends. Biotechnol. 14: 376, 1996) and are nuclease resistant. Alternatively the nuclease resistance for the antisense oligonucleotide can be provided by having a 9 nucleotide loop forming sequence at the 3′-terminus having the nucleotide sequence CGCGAAGCG. The use of avidin-biotin conjugation reaction can also be used for improved protection of AS-ODNs against serum nuclease degradation (see Boado and Pardridge, Bioconj. Chem. 3: 519-23, 1992). According to this concept the antisense oligonucleotide agents are monobiotinylated at their 3′-end. When reacted with avidin, they form tight, nuclease-resistant complexes with 6-fold improved stability over non-conjugated oligonucleotides.

Other studies have shown extension in vivo of antisense-oligodeoxynucleotides (Agrawal et al., Proc. Natl. Acad. Sci. USA 88: 7595, 1991). This process, presumably useful as a scavenging mechanism to remove alien AS-oligonucleotides from the circulation, depends upon the existence of free 3′-termini in the attached oligonucleotides on which the extension occurs. Therefore partial phosphorothioate, loop protection or biotin-avidin at this important position should be sufficient to ensure stability of these antisense-oligodeoxynucleotides.

The present invention also includes use of all analogs of, or modifications to, an oligonucleotide of the invention that does not substantially affect the function of the oligonucleotide or ribozyme. Such substitutions may be selected, for example, in order to increase cellular uptake or for increased nuclease resistance as is known in the art. The term may also refer to oligonucleotides or ribozymes, which contain two or more distinct regions where analogs have been substituted.

The nucleotides can be selected from naturally occurring or synthetically modified bases. Naturally occurring bases include adenine, guanine, cytosine, thymine and uracil. Modified bases of the oligonucleotides include, but are not limited to, xanthine, hypoxanthine, 2-aminoadenine, 6-methyl, 2-propyl and other alkyl adenines, 5-halo uracil, 5-halo cytosine, 6-aza cytosine and 6-aza thymine, pseudo uracil, 4-thiouracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thioalkyl guanines, 8-hydroxyl guanine and other substituted guanines, other aza and deaza adenines, other aza and deaza guanines, 5-trifluoromethyl uracil and 5-trifluoro cytosine.

In addition, analogs of nucleotides can be prepared wherein the structure of the nucleotide is fundamentally altered and that are better suited as therapeutic or experimental reagents. An example of a nucleotide analog is a peptide nucleic acid (PNA) wherein the deoxyribose (or ribose) phosphate backbone in DNA (or RNA) is replaced with a polyamide backbone, which is similar to that found in peptides. PNA analogs have been shown to be resistant to degradation by enzymes and to have extended lives in vivo and in vitro. Further, PNAs have been shown to bind stronger to a complementary DNA sequence than a DNA molecule. This observation is attributed to the lack of charge repulsion between the PNA strand and the DNA strand. Other modifications that can be made to oligonucleotides include polymer backbones, morpholino polymer backbones (see, e.g., U.S. Pat. No. 5,034,506, the contents of which are incorporated herein by reference), cyclic backbones, or acyclic backbones, sugar mimetics or any other modification including which can improve the pharmacodynamics properties of the oligonucleotide.

In some embodiments, DNA enzymes are employed as p16-inhibitory agents to decrease expression of the target p16 mRNA. DNA enzymes incorporate some of the mechanistic features of both antisense and ribozyme technologies. DNA enzymes are designed so that they recognize a particular target nucleic acid sequence, much like an antisense oligonucleotide, however much like a ribozyme they are catalytic and specifically cleave the target nucleic acid.

There are currently two basic types of DNA enzymes, and both of these were identified by Santoro and Joyce (see, for example, U.S. Pat. No. 6,110,462). The 10-23 DNA enzyme comprises a loop structure which connect two arms. The two arms provide specificity by recognizing the particular target nucleic acid sequence while the loop structure provides catalytic function under physiological conditions.

Briefly, to design DNA enzyme that specifically recognizes and cleaves a target nucleic acid, one of skill in the art must first identify the unique target sequence. This can be done using the same approach as outlined for antisense oligonucleotides. In certain instances, the unique or substantially sequence is a G/C rich of approximately 18 to 22 nucleotides. High G/C content helps insure a stronger interaction between the DNA enzyme and the target sequence.

When synthesizing the DNA enzyme, the specific antisense recognition sequence that targets the enzyme to the message is divided so that it comprises the two arms of the DNA enzyme, and the DNA enzyme loop is placed between the two specific arms.

Methods of making and administering DNA enzymes can be found, for example, in U.S. Pat. No. 6,110,462. Similarly, methods of delivery DNA ribozymes in vitro or in vivo include methods of delivery RNA ribozyme, as outlined herein. Additionally, one of skill in the art will recognize that, like antisense oligonucleotides, DNA enzymes can be optionally modified to improve stability and improve resistance to degradation.

The synthetic nuclease resistant antisense oligodeoxynucleotides, ribozymes, etc. of the present invention can be synthesized by any method known in the art. For example, an Applied Biosystems 380B DNA synthesizer can be used. Final purity of the oligonucleotides or ribozymes is determined as is known in the art.

Some embodiments of the invention make use of materials and methods for effecting repression of a target INK4 gene (e.g., p16) by means of RNA interference (RNAi). RNAi is a process of sequence-specific post-transcriptional gene repression that can occur in eukaryotic cells. In general, this process involves degradation of an mRNA of a particular sequence induced by double-stranded RNA (dsRNA) that is homologous to that sequence. For example, the expression of a long dsRNA corresponding to the sequence of a particular single-stranded mRNA (ss mRNA) will labilize that message, thereby “interfering” with expression of the corresponding gene. Accordingly, any selected gene may be repressed by introducing a dsRNA which corresponds to all or a substantial part of the mRNA for that gene. It appears that when a long dsRNA is expressed, it is initially processed by a ribonuclease III into shorter dsRNA oligonucleotides of as few as 21 to 22 base pairs in length. Accordingly, RNAi may be effected by introduction or expression of relatively short homologous dsRNAs. Indeed the use of relatively short homologous dsRNAs may have certain advantages as discussed below.

Mammalian cells have at least two pathways that are affected by double-stranded RNA (dsRNA). In the RNAi (sequence-specific) pathway, the initiating dsRNA is first broken into short interfering (si) RNAs, as described above. The siRNAs have sense and antisense strands of about 21 nucleotides that form approximately 19 nucleotide si RNAs with overhangs of two nucleotides at each 3′ end. Short interfering RNAs are thought to provide the sequence information that allows a specific messenger RNA to be targeted for degradation. In contrast, the nonspecific pathway is triggered by dsRNA of any sequence, as long as it is at least about 30 base pairs in length. The nonspecific effects occur because dsRNA activates two enzymes: PKR (double-stranded RNA-activated protein kinase), which in its active form phosphorylates the translation initiation factor eIF2 to shut down all protein synthesis, and 2′, 5′ oligoadenylate synthetase (2′, 5′-antisense), which synthesizes a molecule that activates RNase L, a nonspecific enzyme that targets all mRNAs. The nonspecific pathway may represent a host response to stress or viral infection, and, in general, the effects of the nonspecific pathway are minimized in particularly useful methods of the present invention. Significantly, longer dsRNAs appear to be required to induce the nonspecific pathway and, accordingly, dsRNAs shorter than about 30 bases pairs are particular useful to effect gene repression by RNAi (see, e.g., Hunter et al., J. Biol. Chem. 250: 409-417, 1975; Manche et al., Mol. Cell Biol. 12: 5239-5248, 1992; Minks et al., J. Biol. Chem. 254: 10180-10183, 1979; and Elbashir et al., Nature 411: 494-8, 2001).

RNAi has been shown to be effective in reducing or eliminating the expression of a target gene in a number of different organisms including Caenorhabditis elegans (see e.g., Fire et al., Nature 391: 806-811, 1998), mouse eggs and embryos (Wianny et al., Nature Cell Biol. 2: 70-75, 2000; and Svoboda et al., Development 127: 4147-4156, 2000), and cultured RAT-1 fibroblasts (Bahramina et al., Mol. Cell Biol. 19: 274-83, 1999), and appears to be an anciently evolved pathway available in eukaryotic plants and animals (Sharp P., Genes Dev. 15: 485-490, 2001).

RNAi has proven to be an effective means of decreasing gene expression in a variety of cell types including HeLa cells, NIH/3T3 cells, COS cells, 293 cells and BHK-21 cells, and typically decreases expression of a gene to lower levels than that achieved using antisense techniques and, indeed, frequently eliminates expression entirely (see, e.g., Bass, Nature 411: 428429, 2001). In mammalian cells, siRNAs are effective at concentrations that are several orders of magnitude below the concentrations typically used in antisense experiments (Elbashir et al., Nature 411: 494-498, 2001).

Certain double stranded oligonucleotides used to effect RNAi are less than 30 base pairs in length and may comprise about 25, 24, 23, 22, 21, 20, 19, 18 or 17 base pairs of ribonucleic acid. Optionally, the dsRNA oligonucleotides of the invention may include 3′ overhang ends. Non-limiting exemplary 2-nucleotide 3′ overhangs may be composed of ribonucleotide residues of any type and may even be composed of 2′-deoxythymidine resides, which lowers the cost of RNA synthesis and may enhance nuclease resistance of siRNAs in the cell culture medium and within transfected cells (see Elbashi et al., Nature 411: 494498, 2001).

Longer dsRNAs of 50, 75, 100 or even 500 base pairs or more may also be utilized in certain embodiments of the invention. Exemplary concentrations of dsRNAs for effecting RNAi are about 0.05 nM, 0.1 nM, 0.5 nM, 1.0 nM, 1.5 nM, 25 nM or 100 nM, although other concentrations may be utilized depending upon the nature of the cells treated, the gene target and other factors readily discemable the skilled artisan. Exemplary dsRNAs may be synthesized chemically or produced in vitro or in vivo using appropriate expression vectors. Exemplary synthetic RNAs include 21 nucleotide RNAs chemically synthesized using methods known in the art (e.g., Expedite RNA phophoramidites and thymidine phosphoramidite (Proligo, Germany)). Synthetic oligonucleotides may be deprotected and gel-purified using methods known in the art (see e.g., Elbashir et al., Genes Dev. 15: 188-200, 2001). Longer RNAs may be transcribed from promoters, such as T7 RNA polymerase promoters, known in the art. A single RNA target, placed in both possible orientations downstream of an in vitro promoter, will transcribe both strands of the target to create a dsRNA oligonucleotide of the desired target sequence.

The specific sequence utilized in design of the oligonucleotides may be any contiguous sequence of nucleotides contained within the expressed gene message of the target. Programs and algorithms, known in the art, may be used to select appropriate target sequences. In addition, optimal sequences may be selected, as described additionally above, utilizing programs designed to predict the secondary structure of a specified single stranded nucleic acid sequence and allow selection of those sequences likely to occur in exposed single stranded regions of a folded mRNA. Methods and compositions for designing appropriate oligonucleotides may be found in, for example, U.S. Pat. No. 6,251,588, the contents of which are incorporated herein by reference. mRNA is generally thought of as a linear molecule that contains the information for directing protein synthesis within the sequence of ribonucleotides. However, studies have revealed a number of secondary and tertiary structures exist in most mRNAs. Secondary structure elements in RNA are formed largely by Watson-Crick type interactions between different regions of the same RNA molecule. Important secondary structural elements include intramolecular double stranded regions, hairpin loops, bulges in duplex RNA and internal loops. Tertiary structural elements are formed when secondary structural elements come in contact with each other or with single stranded regions to produce a more complex three-dimensional structure. A number of researchers have measured the binding energies of a large number of RNA duplex structures and have derived a set of rules which can be used to predict the secondary structure of RNA (see e.g., Jaeger et al., Proc. Natl. Acad. Sci. (USA) 86: 7706, 1989; and Turner et al., Ann. Rev. Biophys. Biophys. Chem. 17: 167, 1988). The rules are useful in identification of RNA structural elements and, in particular, for identifying single stranded RNA regions, which may represent preferred segments of the mRNA to target for silencing RNAi, ribozyme or antisense technologies. Accordingly, particular segments of the mRNA target can be identified for design of the RNAi mediating dsRNA oligonucleotides as well as for design of appropriate ribozyme and hammerhead ribozyme compositions of the invention.

The dsRNA oligonucleotides may be introduced into the cell by transfection using carrier compositions such as liposomes, which are known in the art, e.g., Lipofectamine 2000 (Life Technologies, Rockville Md.) as described by the manufacturer for adherent cell lines. Transfection of dsRNA oligonucleotides for targeting endogenous genes may be carried out using Oligofectamine (Life Technologies). Transfection efficiency may be checked using fluorescence microscopy for mammalian cell lines after co-transfection of hGFP encoding pAD3 (Kehlenback et al., J. Cell. Biol. 141: 863-74, 1998). The effectiveness of the RNAi may be assessed by any of a number of assays following introduction of the dsRNAs- These include, but are not limited to, Western blot analysis using antibodies which recognize the targeted gene product following sufficient time for turnover of the endogenous pool after new protein synthesis is repressed, and Northern blot analysis to determine the level of existing target mRNA.

Still further compositions, methods and applications of RNAi technology for use in the invention are provided in U.S. Pat. Nos. 6,278,039; 5,723,750; and 5,244,805, which are incorporated herein by reference.

Thus, the invention provides therapeutic compositions for use in treating cancer patients bearing adriamycin-resistant neoplastic cells. These therapeutic compositions comprise an agent that inhibits p16 expression and a pharmaceutically acceptable carrier. A “pharmaceutically acceptable carrier” includes, without limitation, any and all solvents, dispersion media, coatings, antibacterial and antifungal agents, isotonic and absorption-delaying agents, and agents which improve composition internalization by a cell which are non-toxic to the cell, and which do not reduce the therapeutic activity of the agent that inhibits p16 expression. Except insofar as any conventional medium or agent is incompatible with the active ingredient, its use as a pharmaceutically acceptable carrier in the therapeutic compositions of the invention is contemplated. Methods for making pharmaceutically acceptable carriers and formulations thereof are found, for example, in Remington's Pharmaceutical Sciences (18^{th} Ed.), ed. A. Gennaro, Mack Publishing Company, Easton, Pa. 1990; and in Remington: The Science and Practice of Pharmacy (20^{th} Ed.), ed. A. Gennaro, Lippincott Williams & Wilkins, Philadelphia, Pa. 2003.

The agent that inhibits p16 expression may be combined with pharmaceutically acceptable carriers to generate therapeutic compositions for use in vivo. Accordingly, the p16-inhibiting agent of the invention may be formulated for administration with pharmaceutically acceptable carriers such as water, buffered saline, polyol (e.g., glycerol, propylene glycol, liquid polyethylene glycol), or suitable mixtures thereof. In one embodiment, an agent that inhibits p16 is dispersed in liquid formulations, such as micelles or liposomes, which closely resemble the lipid composition of natural cell membranes. Formulations for parenteral administration may, for example, contain sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-poloxypropylene copolymers may be used to control the release of the agent that inhibits p16 expression. Other potentially useful parenteral delivery systems include ethylene-vinyl acetate copolymer particles, osmotic pumps, implantable infusion system, and liposomes.

Any route of administration may be used to administer the agent that inhibits p16 of the invention. Accordingly, non-limiting routes by which the agent that inhibits p16 may be administered include, for example, intravenous, intraperitoneal, oral, subcutaneous, intradermal, intramuscular, intradermal, topical, oral, rectal, intra-ocular, and intra-nasal (e.g., by aerosol). Of course, the pharmaceutically acceptable carrier chosen to accompany the p16-inhibitory agent of the invention may differ depending upon which route of administration is used.

One non-limiting advantage for using an agent that inhibits p16 (e.g., a p16 siRNA) for reversing ADR drug resistant in solid tumors is that the p16 siRNAs can be used in combination with well known anticancer drugs, such as adriamycin and other anthracyclines (e.g. daunorubicin, epirubicin, idarubicin), that are that are well characterized, effective, and currently used in the clinic. Of further interest may be their use in combination with other agents that have been designed to inhibit gene expression of other important genes involved in drug resistance, such as p-glycoprotein siRNAs.

Thus, in certain embodiments, the composition of the invention comprising an agent that inhibits p16 and a pharmaceutically acceptable carrier also comprises adriamycin or another chemotherapeutic agent (e.g., another anthracycline). In accordance with this aspect of the invention, the p16-inhibitory agent and adriamycin may be administered to the same neoplastic cell in the patient, such that the p16-inhibitory agent acts to make the cell more sensitive to adriamycin.

Further, the invention provides a method for treating and/or relieving the symptoms of a cancer patient comprising administering the patient an agent that inhibits p16 and adriamycin or another chemotherapeutic agent (e.g., another anthracycline). In some embodiments, the patient is also administered a pharmaceutically acceptable carrier.

In some embodiments, the agent that inhibits p16 and the adriamycin are each administered in a therapeutically effective amount. As used herein, the term “therapeutically effective amount” means the total amount of each active component of a therapeutic composition that is sufficient to show a meaningful patient benefit. When administered to an animal having a solid tumor, a therapeutically effective amount is an amount sufficient to slow tumor growth, or to arrest tumor growth, or to diminish tumor size. Where the neoplasm is a non-hematologoical non-solid tumor, the neoplastic cells may be counted, and a therapeutically effective amount of the compositions of the invention will slow the increase in number of neoplastic cells, or prevent an increase in the number of neoplastic cells, or reduce the number of neoplastic cells.

When applied to an individual active component, administered alone (e.g., the p16 expression-inhibiting agent alone), a therapeutically effective amount refers to that component alone. When applied in combination (e.g., the combination of the p16 expression-inhibiting agent with adriamycin), the term refers to the combined amounts of the active components that result in the therapeutic effect, whether the components are administered in combination, serially, or simultaneously. What constitutes a therapeutically effective amount is within the skill of one of ordinarily skill, and can be readily determined in non-human mammals prior to use in human patients. In one non-limiting example of a therapeutically effective amount, where the p16 expression-inhibiting agent is administered directly into a solid tumor of approximately 40 mm³, approximately 5 μg-50 μg of p16 siRNA is administered per day for 3 days. A larger amount of p16 expression-inhibiting agent and/or administration for more than 3 days is used for solid tumors larger than approximately 40 mm³.

The following examples are intended to further illustrate certain preferred embodiments of the invention and are not limiting in nature. Those skilled in the art will recognize, or be able to ascertain, using no more than routine experimentation, numerous equivalents to the specific substances and procedures described herein.

### EXAMPLE 1

Differential P16 Expression in Adriamycin-Resistant Tumor Cells

Two-dimensional gel analysis, followed by mass spectroscopy, was performed to determine which proteins were differentially expressed by adriamycin-resistant cells and their adriamycin-sensitive counterparts.

For these studies, breast adenocarcinoma cell lines MCF7 and MDA-MB-231 were obtained from American Type Culture Collection (“ATCC”; Manassas, Va., USA). Drug resistant MCR7/AR cells (derived from drug-sensitive MCF7 cells), which are ten times more resistant to adriamycin than its drug-sensitive parent cell line, were provided by McGill University, Montreal, Quebec, Canada. Drug resistant MDA-MB-231/AR cells (derived from drug-sensitive MDA-MB-231 cells), which are ten times more resistant to adriamycin than its drug-sensitive parent cell line, were provided by Aurelium BioPharma Inc., (Montreal, Quebec, Canada).

Cell culture supplies were purchased from Gibco Life Technologies (Burlington, Ont., Canada). Adriamycin (ADR, also called doxorubicin) was purchased from Sigma Chemical Co. (St. Louis, Mo., USA) and stored according to the supplier's recommendations. MCF7 and MCR7/AR cells were cultured in αMEM medium supplemented with 10% fetal bovine serum (FBS). MDA-MB-231 and MDA-MB-231/AR cells were cultured in DMEM high glucose medium+10% FBS.

All culture media contained glutamine (gln) at 2 mM final concentration, and all cells were cultured at 37° C. in a humid atmosphere containing 5% CO₂. Drug-sensitive MCF7 and MDA-MB-231 were grown in the presence of adriamycin, while MCF7/AR and MDA-MB-231/AR were grown continuously in the presence of 4.8 μM or 0.4 μM adriamycin, respectively. Cell lines were routinely tested for mycoplasma using a PCR-based mycoplasma detection kit (commercially available from Stratagene Inc., San Diego, Calif., USA, according to manufacturer's protocol) and tested negative for mycoplasma.

Total cell protein lysates (also called extracts) were prepared according to standard methods (all reagents were from Sigma Chemical Co, St. Louis, Mo.). Briefly, cultured cells were rinsed 2 times with 15 mL PBS and incubated in 15 mL of Cell Dissociation Buffer (commercially available from Sigma Chemical Co.) at 37° C. for approximately 10 minutes (i.e., or until they detached from the flask). Cells were collected in a 15 mL tube and centrifuged for 5 minutes at room temperature (RT) at 1,000 rpm (800×g). The supernatant was discarded and cells were washed 3 times with PBS at RT. The cell pellet was transferred to a microtube and 500 μL of PBS was added. The cells were centrifuged 5 minutes at 3,000 rpm in an Eppendorf Microfuge. The supernatant was removed by decanting and the pelleted cells were lysed in 50-150 μL of lysis buffer (50 mM NaCl, 50 mM Tris pH 8 and 4% CHAPS), containing 1 μg/mL each of the protease inhibitors pepstatin, leupeptin, and benzamidine, and 0.2 mM PMSF), and incubated 5 minutes on ice. The cell lysates were then sonicated with a Vibracell sonicator set at amplitude 40 setting #25 for 3 times 10 seconds with 1 minute incubations on ice between sonications, and stored at −80° C.

For two-dimensional (2D) gel electrophoresis, total cell lysates (also referred to as extracts) were thawed and then incubated with 1 U/μL DNAse I and 5 mM MgCl₂ (final concentrations) for 2 hrs on ice. The protein concentration of each lysate was determined using the RC DC protein assay kit, according to the manufacturer's instructions (BioRad Laboratories, Hercules, Calif., USA; see also Lowry et al., J. Biol. Chem. 193: 265-275, 1951). Finally, urea powder (commercially available from J. T. Baker Co., a division of Mallinckrodt Baker, Inc., Phillipsburg, N.J.) was added to the cell lysates to a final concentration of 8 M. Equivalent amounts of protein (250 μg) from total cell extracts from drug-sensitive cell types (e.g., MCF7 or MDA-MB-231) and multidrug-resistant cell types (e.g., MCF7/AR and MDA-MB-231/AR) were analyzed by 2D gel electrophoresis and visualized by silver staining. For the first dimension, isoelectric focusing (IEF) was achieved using immobilized pH gradient gel (IPG) strips (pH 4-7, 24 cm, Amersham Pharmacia Biotech, Piscataway, N.J., USA), according to the manufacturers recommendations.

For the second dimension, the above isoelectric strips were subject to sodium dodecyl sulfate-polyacrylamide gel electrophoresis (SDS-PAGE) using 12.5% gels, according to the method of Laemmli (Laemmli U. K., Nature 227:680-685, 1970). Molecular weight markers were loaded onto a 2×3 mm filter paper and placed at one end of the strip. The strip and molecular weight marker filter were then sealed onto the polyacrylamide gel with a 0.5% agarose solution in running buffer. The gels were run at constant current (5 mA/gel) for 30 minutes, and the current was then increased to 10 mA/gel for 6 hours.

The 2D gels were fixed in a 40% (v/v) methanol: 10% (v/v) acid acetic solution for 24 hours at room temperature and then silver stained according to standard methods (see, e.g., Ausubel et al., Current Protocols in Molecular Biology, John Wiley & Sons, New York, N.Y. 1988-2004, updated yearly). The 2D maps (proteomes) of total cell extracts were compared by using ImageMaster 2D Elite software (Amercham Pharmacia Biotech) and checked manually.

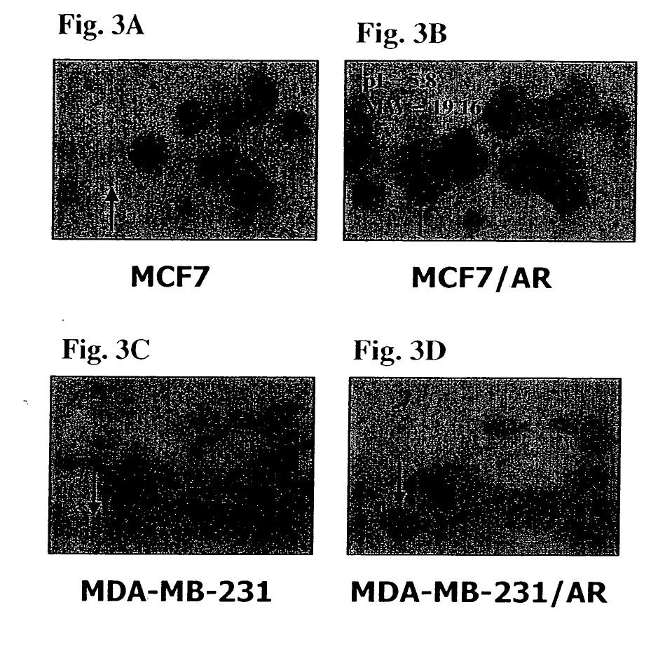

FIGS. 3A-3D show the localization of p16^{INK4a} protein spots on 2D gels of extracts of ADR-resistant and sensitive cells with anti-p16 monoclonal antibody in two ADR-sensitive human breast tumor cell lines and their counterpart ADR-resistant lines. Approximately equivalent amounts of each of the cell extracts were resolved on SDS-PAGE and transferred to nitrocellulose membrane. The membranes were probed with p16INK4a specific monoclonal antibody (α-p16INK4a (Ab4, clone 16P04, JC2; Neomarkers). As noted in FIG. 3B, the p16 spot migrates with a pI of 5.8 and has a molecular mass of 19.16.

Cell lines MCR7 (FIG. 3A) and MDA-MB-231 (FIG. 3C) are ADR-sensitive to approximately 80 and approximately 200 nM ADR, respectively, whereas their selected derivative cell lines MCF7/AR (FIG. 3B) and MDA-MB-231/AR (FIG. 3D) are resistant to 4.8 μM and 0.4 μM ADR respectively. As can be seen in FIGS. 3A-3B, p16 protein was present in significantly higher amounts (10-20 fold higher) in the adriamycin resistance human breast cell line MCF7/AR (FIG. 3B, spot indicated by the arrow) than in the drug-sensitive parent cell line MCF7 (FIG. 3A, spot indicated by the arrow). Similarly, p16 protein was present in approximately 5 fold higher amounts in adriamycin-resistant MDA-MB-231/AR cells (FIG. 3D, spot indicated by the arrow) than in the drug-sensitive parental cell line MDA-MB-231 (FIG. 3C, spot indicated by arrow).

The level of P-glycoprotein mRNA was also determined for these cell lines by microarray analysis.

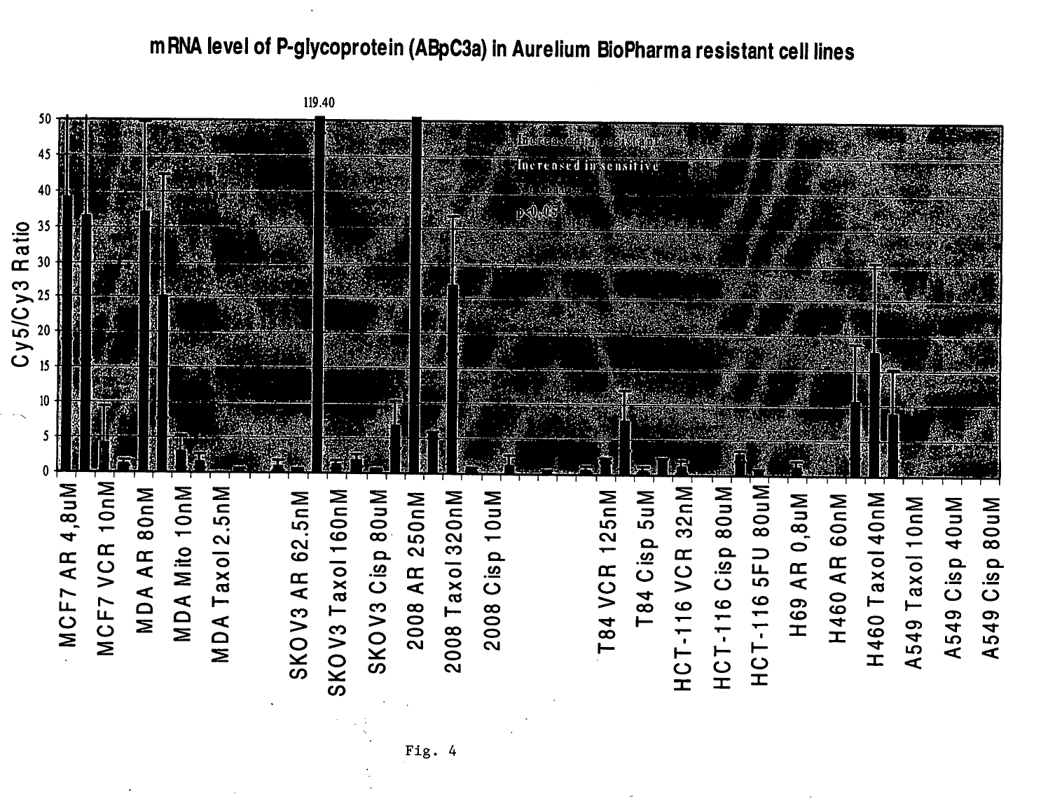

As shown in FIG. 4, the level of p-glycoprotein mRNA was found to be increased approximately 52 fold and approximately 40 fold in the ADR-resistant cell lines (MDA-MB-231/AR and MCF7/AR, respectively), as compared to MDA-MB-231 and MCF7, respectively. Note that although MCF7/AR is ten fold more resistant to adriamycin than MDA/AR, the expression of P-glycoprotein is lower in MCF7/AR cells than in MDA/AR. In other words, there is no direct correlation between expression of P-glycoprotein and resistance to adriamycin across different cell types.

Next, mass spectrometry samples were prepared. To do this, the identity of the spot indicated by the arrow as p16^{INK4a} in MCF7/AR cells (from FIG. 3B) was excised with a clean (acid washed) razor blade, cut into small pieces on a clean glass plate, and transferred to a 200 μl PCR tube (MeOH treated). To remove the silver strain, the gel pieces were mixed with 50 μl destainer A and 50 μl destainer B (provided with SilverQuest kit, Life Technologies) (or 100 μl of the destainers A and B were premixed and used immediately) and incubated for 15 min at room temperature without agitation. The destaining solution was removed using a capillary tip. Water (200 μL) was added to the gel pieces to remove the destaining solution, and the tubes were vortexed and incubated 10 minutes at room temperature. The latter step was repeated three times. The gel pieces were then dehydrated in 100 μl 100% methanol for 5 minutes at room temperature, followed by rehydration in 30% methanol/water for 5 minutes. Gel pieces were then washed 2 times in water for 10 minutes and 2 times in an Ambic A solution (25 mM ammonium bicarbonate+30% (v/v) acetonitrile) for 10 min minutes. The gel pieces were completely dried in a speed vac for 20 minutes, and then subjected to tryptic digestion by adding approximately 1 volume of trypsin solution (130 ng trypsin (Roche Diagnostics, Laval, Qc, Canada) in Ambic B solution (25 mM ammonium bicarbonate+5 mM CaCl₂; also called digestion buffer) to 1 volume of gel piece and the gel samples incubated on ice for 45 minutes. Fresh digestion buffer (10 μl) was added a second time and digestion allowed to proceed for a further 15-16 hours at 37° C.

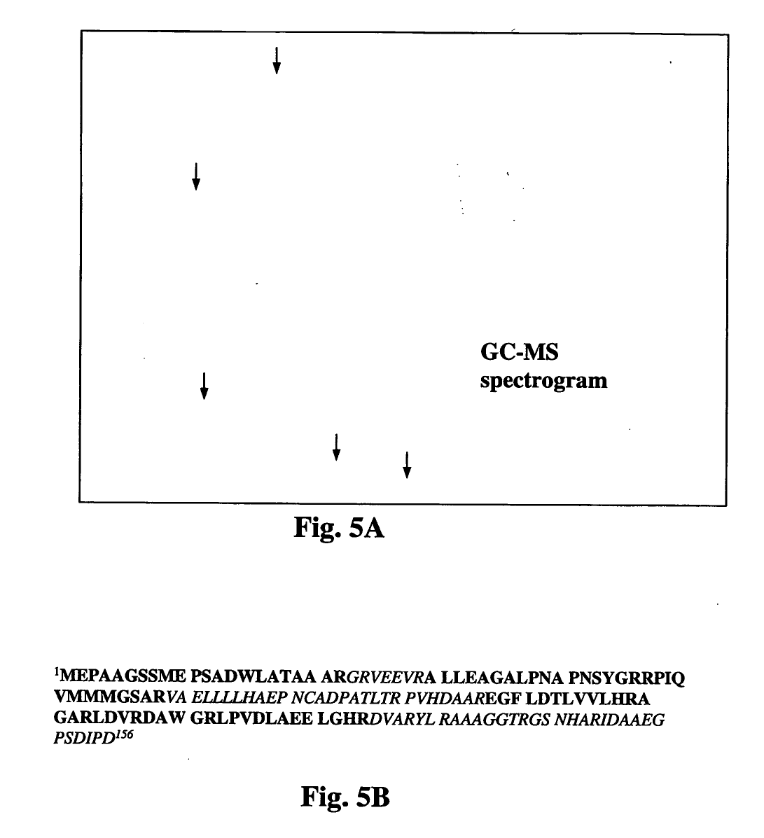

Trypsin-digested peptides were extracted with 20 μL acetonitrile for 15 minutes at room temperature with shaking. The gel pieces/solvent were sonicated 5 minute and reextracted with 50 μL of a freshly prepared solution of 5% formic acid:50% acetonitrile:45% water. The extraction step was repeated several times and the collected material combined and lyophilized to dryness. The extracted peptides were resuspended in 5% methanol containing 0.2% trifluroacetic acid, and then loaded onto an equilibrated C18 bed (Ziptip from Millipore, Bedford, Mass., USA). The loaded Ziptip was washed with 5% acetonitrile containing 0.2% TFA and then eluted in 10 μl of 60% acetonitrile. Eluted peptide solution was dried and analyzed using MALDI mass spectroscopy (Mann M. et al., Ann. Rev. Biochem. 70: 437-473, 2001) (see FIG. 5A) and MALDI-TOF-MS software.

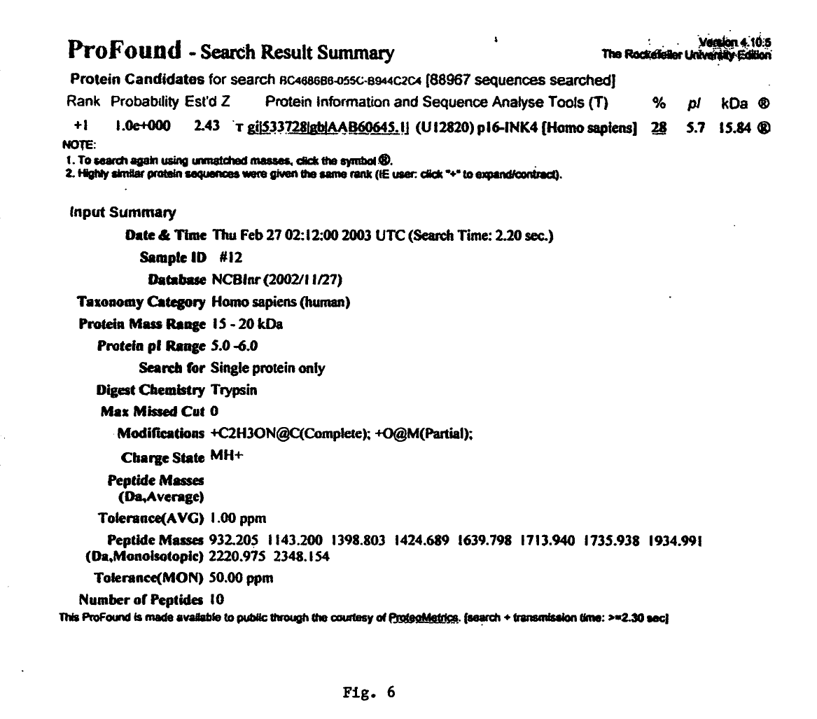

The resulting peptide sequences were further analyzed using the sequence database search shareware software program ProFound™ (http://www.proteomics.com/prowl-cgi/Profound.exe) to obtain protein identity. FIG. 6 shows the read-out from the PROFOUND search. PROFOUND was used to search public databases for protein sequences (e.g., non-redundant collection of sequences at the US National Center for Biotechnology Information (NCBInr)). The NCBInr database contains translated protein sequences from the entire collection of DNA sequences kept at GenBank, and also the protein sequences in the PDB, SWISS-PROT and PIR databases.

FIG. 5B, FIG. 6, and Table I show the identification of the 2D-gel spot overexpressed in adriamycin-resistant MCF7/AR breast tumor cells as P16 Mike Egan INK4a by MALDI-TOF-MS with approximately 100% certainty. The 2D-gel spot mass spectroscopy fingerprint is presented in FIG. 5A with the major tryptic peptides indicated by arrows. The amount of amino acid sequence obtained by this analysis was 81 amino acids or 52% of the entire 156 amino acids of p16^{INK4a} protein. Table I provides the peptides sequences obtained by GC-MS analysis, with the amino acids represented by standard single-letter code. Note that peptides 4 and 6 sequences overlap whereas the remaining peptide sequences are unique.

TABLE I

Tryptic peptide sequences of the protein overexpressed in
adriamycin-resistant human breast tumor MCF7/AR cells, as
determined by MALDI-TOF MS.

| | |
|---|---|
| Mass | Mass |

| | | | |
|---|---|---|---|
| Peptides | submitted | matched | Peptide sequence |

| | | | |
|---|---|---|---|
| 1 | 1398.803 | 1398.774 | ⁸⁸EGFLDTLVVLHR⁹⁹ |
| 2 | 1424.689 | 1424.681 | ⁴⁷RPIQVMMMGSAR⁵⁸ |
| 3 | 1713.940 | 1713.892 | ³⁰ALLEAGALPNAPNSYGR⁴⁶ |
| 4 | 1934.991 | 1933.988 | ¹⁰⁸DAWGRLPVDLAEELGHR¹²⁴ |
| 5 | 2220.975 | 2220.006 | ¹MEPAAGSSMEPSADWLATAAAR²² |
| 6 | 2220.975 | 2222.204 | ¹¹³LPVDLAEELGHERDARYLR¹³¹ |

The peptide sequences shown in Table I were analyzed using the ProFound program and determined to correspond to sequences of human p16^{INK4a}. The sequence data obtained in this analysis was sufficient to identify the 2D-gel protein spot as p16^{INK4a} protein with aprox. 100% certainty.

The alignment of the sequences of the tryptic peptides with the p16INK4a protein sequence is shown in FIG. 5B. The full length sequence of p16INK4a protein is provided in SEQ ID NO: 1, and is also available as GenBank Accession No. AB060808.1; GI:20330501.

FIG. 6 shows the results of the ProFound software search results summary of the GC-MS data.

These results demonstrate with certainty that the p16 protein is overexpressed in adriamycin-resistant cells as compared to their adriamycin-sensitive counterparts.

### EXAMPLE 2

Adriamycin Sensitivity in Tumor Cells

Experiments were next performed to determine whether various tumor cells (both solid tumor and hematological tumors) having different sensitivity to adriamycin had different levels of p16^{INK4a} expression.

For these studies, the following human cell lines were obtained from American Type Culture Collection (“ATCC”; Manassas, Va., USA). These included breast adenocarcinoma cell lines MCF7 and MDA-MB-231, ovarian adenocarcinoma cell lines SKOV3 and OVCAR3, prostate adenocarcinoma cell line PC3, acute lymphoblastic leukemia cell lines CEM and MOLT-4, chronic myelogeneous leukemia cell line K-562 and acute promyelocytic leukemia HL60. The drug resistant cells, MCF7/AR, SKOV3/VLB, small cell lung carcinoma H69, H69/AR, HL60/AR, CEM/VLB 0.1 μM and CEM/VLB 1 μM, were provided by McGill University, Montreal, Quebec, Canada. Other drug resistant cells (namely, MCF7/VLB, MCF7/VCR, MCF7/Mito, MDA-MB-231/AR, MDA-MB-231/Mito, SKOV3/CIS, SKOV3/Taxol, PC3/Melphalan, CEM/AR 0.8 μM, CEM/AR 10 μM, MOLT4/VLB 25 nM, MOLT4/AR 250 nM and MOLT4/AR 500 nM) were provided by Aurelium BioPharma Inc., (Montreal, Quebec, Canada). These cell lines were derived by a series of stepwise selection by culturing the cells in increasing drug concentrations. The drug resistant cell lines derived from a non-resistant cell line share part of the name of that cell line. For example, the CEM/AR 0.8 μM, CEM/AR 10 μM cell lines were derived from CEM.

Cell culture supplies were from Gibco Life Technologies (Burlington, Ont., Canada). Cytocidal drugs (vincristin (VCR), vinblastin (VBL), melphalan (mel), taxol, mitoxantrone (mito), and adriamycin (ADR, doxorubicin) were purchased from Sigma Chemical Co. (St. Louis, Mo., USA) and stored according to the supplier's recommendations. Cells were cultured in the following media: (a) αMEM medium supplemented either with 10% fetal bovine serum (FBS) (MCF7 and derivatives, CEM and derivatives) or with 15% FBS (SKOV3 and derivatives), (b) DMEM high glucose medium+10% FBS (MDA-MB-231 and derivatives), (c) RPMI 1640 medium+10 mM HEPES, 1 mM sodium pyruvate, 4.5 g/L glucose, 0.01 mg/mL bovine insulin, and 20% FBS (OVCAR3), (d) Ham's F12 medium (Fl2K)+10% FBS (PC3, PC3/melphalan), (e) RPMI 1640 medium+4 mM L-glutamine (gln) and 10% FBS (H69, H69/AR), (f) RPMI 1640 medium+10 mM HEPES, 1 mM sodium pyruvate, 4.5 g/L glucose and 10% FBS (MOLT4 and derivatives), or (g) RPMI 1640+20 mM HEPES, 1 mM sodium pyruvate and 10% FBS (K-562) and in RPMI 1640 medium+10% FBS (HL60 and HL60/AR).

All culture media contained glutamine (Gln) at 2 mM final concentration, except for H69 and H69/AR cells, which were cultured in culture media containing 4 mM Gln. The drug sensitive cell lines were grown in the absence of antibiotics whereas the drug resistant cell lines were grown in the presence of the appropriate antibiotics. All cells were cultured at 37° C. in a humid atmosphere containing 5% CO₂.

Multidrug resistant cells (MCF7/AR, MCF7/VLB, MCF7/VCR, MCF7/Mito, MDA-MB-231/AR, MDA-MB-231/Mito, SKOV3NLB, SKOV3/CIS, SKOV3/Taxol, PC3/Melphalan, HL60/AR, CEM/VLB 0.1 μM, CEM/VLB 1 μM, CEM/AR 0.8 μM, CEM/AR 10 μM, MOLT4/VLB 25 nM, MOLT4/AR 250 nM and MOLT4/AR 500 nM) were grown continuously with the appropriate concentrations of combinations of cytotoxic drugs. All multidrug resistant cell lines were routinely tested for multidrug resistance using a panel of different cytotoxic drugs representing different drug classes.

Cell lines were routinely tested for mycoplasma using a PCR-based mycoplasma detection kit (commercially available from Stratagene Inc., San Diego, Calif., USA, according to manufacturer's protocol) and tested negative for mycoplasma.

Initially, the cells are tested in a Standardized Adriamycin Resistance Test (“SART”) to determine their resistance to adriamycin. For this test, cells of each tumor cell line are obtained in exponential phase of growth and plated in triplicate at Y cells per x sized plate along with a control cell lines (sensitive to ADR) and an experimental cell line (resistant to ADR). The cells are allowed to attach to the plate overnight and then the media is replaced with media containing an different concentrations of ADR (0 nM, 10 nM, 20 nM, 50 nM, 100 nM, 400 nM, 500 nM, 1 μM, 5 μM, and 10 μM) and the cells are then incubated at 37° C. for 4 days. After this time, the media is decanted and the cells are stained with methylene blue. Cell growth is measured by absorbance at 660 nm. The colonies are the counted and the results expressed as the level of ADR resistance in nM (e.g., ≦400 nM), or EC50, in which 50% of the cells were still alive.

Next, the cell lines were subjected to a Standardized Immunoblot Test for expression of p16 Protein (“SITp16”). For this test, total cell protein lysates (also called extracts) were prepared according to standard methods (all reagents were from Sigma Chemical Co, St. Louis, Mo.). Briefly, cultured cells were rinsed 2 times with 15 mL PBS and incubated in 15 mL of Cell Dissociation Buffer (available from Sigma Chemical Co, St. Louis, Mo.) at 37° C. for approximately 10 minutes (i.e., or until they detached from the flask). Cells were collected in a 15 mL tube and centrifuged for 5 minutes at room temperature (RT) at 1,000 rpm (800×g). The supernatant was discarded and cells were washed 3 times with PBS at RT. The cell pellet was transferred to a microtube and 500 μL of PBS was added. The cells were centrifuged 5 minutes at 3,000 rpm in an Eppendorf Microfuge. The supernatant was removed by decanting and the pelleted cells were lysed in 50-150 μL of lysis buffer (50 mM NaCl, 50 mM Tris, pH 8 and 4% CHAPS), containing 1 μg/mL each of the proteases inhibitors pepstatin, leupeptin, and benzamidine, and 0.2 mM PMSF), and incubated 5 minutes on ice. The cell lysates were then sonicated with a Vibracell sonicator set at amplitude 40 setting #25 for 3 times 10 seconds with 1 minute incubations on ice between sonications, and stored at −80° C.

The protein concentration of each lysate was determined using the RC DC protein assay kit, according to the manufacturer's instructions (BioRad Laboratories, Hercules, Calif., USA; see also Lowry et al., J. Biol. Chem. 193: 265-275, 1951).

Total cell lysates were thawed, and 100 μg of protein (completed to 50 μL with nanopure water) were mixed with 10 μL of 5× electrophoresis buffer (60 mM Tris/HCl pH 6.8, 25% glycerol, 2% SDS, 14.4 mM β-mercaptoethanol, 0.1 % bromophenol blue) and the samples were heated at 100° C. for 5 minutes and loaded onto 10% SDS-PAGE gels. Resolved proteins were electrophoretically transferred onto nitrocellulose membranes (Hybond, Amercham Pharmacia Biotech) for 2 hours. After blocking the membranes with 5% non-fat milk in PBS overnight at 4° C., primary and secondary antibody incubations were in the same buffer at room temperature for 2 hour and 1 hour, respectively. The HRP substrate used was Supersignal West Pico Chemiluminescent Substrate (Pierce, Rockford, Ill., USA). Monoclonal antibody against p16INK4a (Ab-4, clone JC2) was purchased from Neomarkers (Fremont, Calif., USA) and used at 1:× dilution. Control antibodies included anti-pan-actin (Neomarkers: Ab-5, clone ACTN05, C4) and/or anti-bcl-2 monoclonal antibody (NeoMarkers, Ab-1, Clone 10).

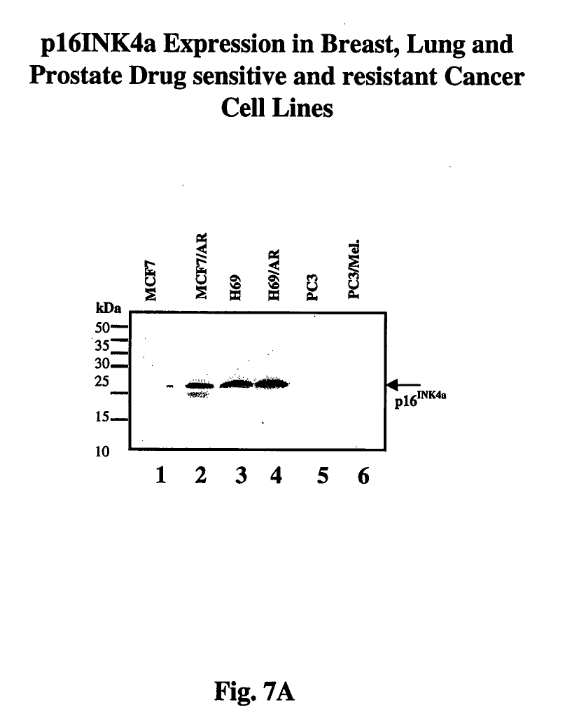

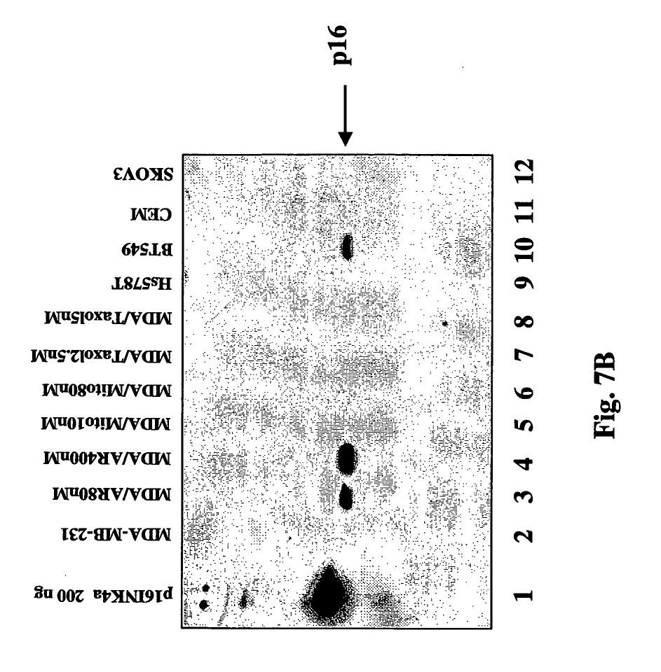

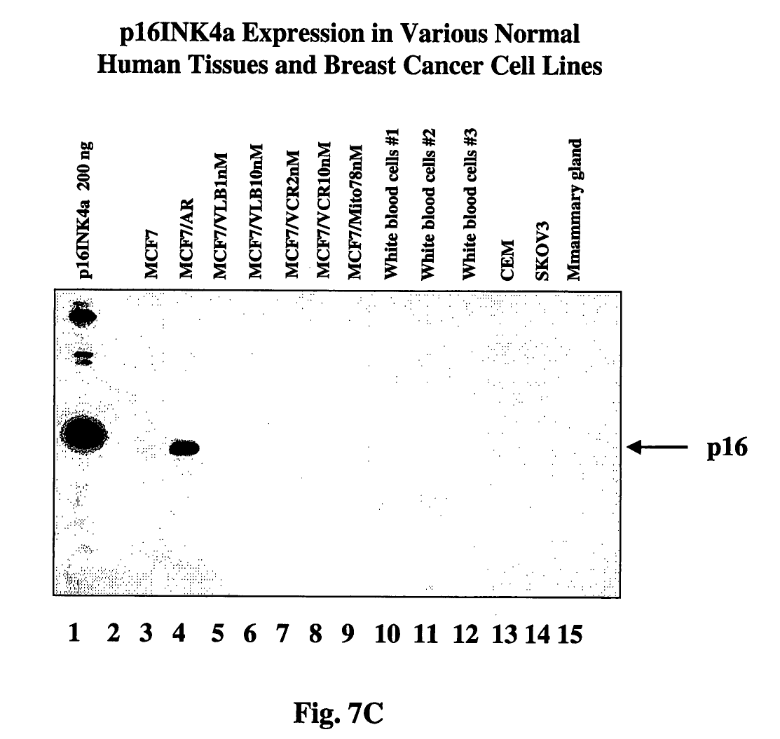

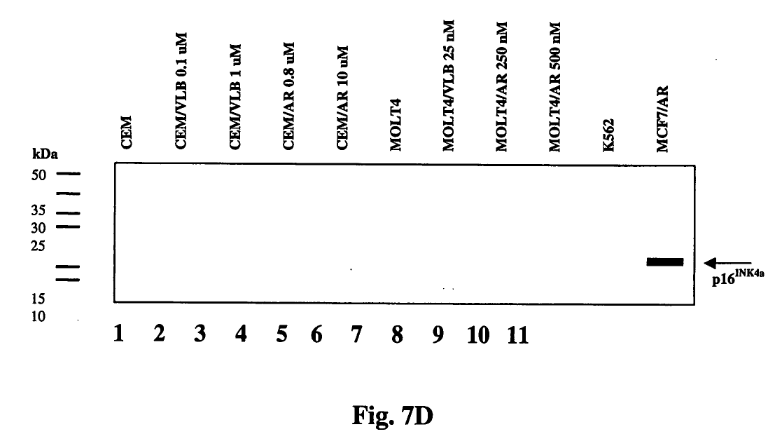

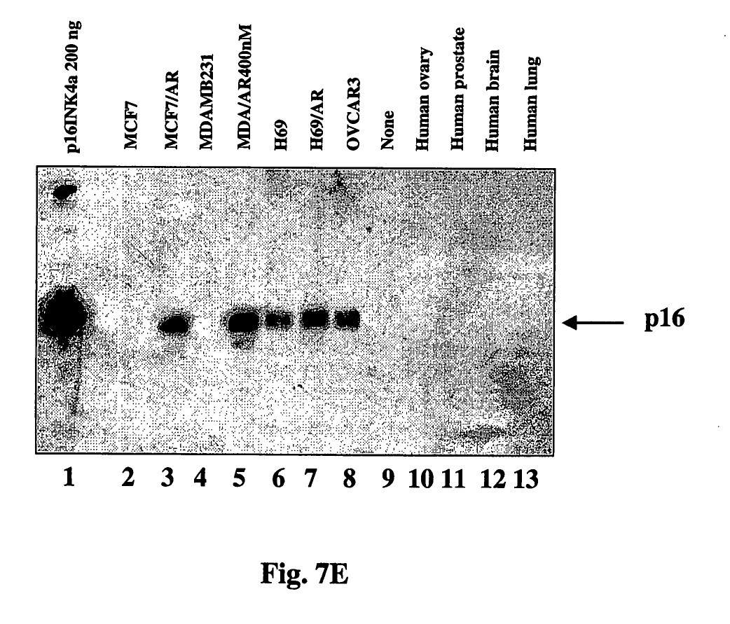

Examples of the SITp16 test results are shown in FIGS. 7A-7E. For these studies, total cells extracts were prepared (as described above) from: (a) normal human breast cells (Hs574Mg, Hs578Bst), (b) a panel of drug-sensitive human tumor cell lines originating from both solid and hematological tumors (Hs574T, Hs578T, MCF7, MDA-MB-231, H69, PC3, SKOV3, HL60, CEM, MOLT4, and K562), (c) and various drug-resistant human cell lines obtained from the drug-sensitive cell lines listed in (b) above (MCF7/AR, MCF7NLB, MCF7/VCR, MCF7/Mito, MDA-MB-231/AR, MDA-MB-231/Mito, H69/AR, PC3/Mel, SKOV3/VLB, HL60/AR, CEM/VLB, CEM/AR, MOTL4/VLB, and MOLT4/AR).

Approximately equivalent amounts of each of the cell extracts were resolved on SDS-PAGE and transferred to nitrocellulose membrane. The membranes were probed with p16INK4a specific monoclonal antibody (α-p16INK4a (Ab-4, clone 16P04, also known as JC2; commercially available from Neomarkers, Inc., exclusively distributed by LabVision Corp., Fremont, Calif.).

FIG. 7A shows the p16 expression in MCF7 cells, MCF7/AR cells (drug sensitive and adriamycin resistant breast tumor cells), H69 cells, H69/AR cells (adriamycin resistant tumor cells), PC3 cells, and PC3/Mel cells (melphalan resistant prostate tumor cells).

FIG. 7B shows the p16 expression in MDA-MB-231 cells, MDA-MB-231/AR80 cells (resistant to 80 nM adriamycin), MDA-MB-231/AR400 cells (resistant to 400 nM adriamycin), MDA-MB-231/Mito 10 nM cells (resistant to 10 nM mitoxantrone), MDA-MB-231/Mito 80 nM cells (resistant to 80 nM mitoxantrone), MDA-MB-231/Taxo 2.5nM cells (resistant to 2.5 nM taxol), MDA-MB-231 5 nM cells (resistant to 5 nM taxol), Hs578T cells, BT549 cells (breast tumor cells), CEM cells (lymphoid T-cell leukemia), and SKOV3 cells (ovarian cancer cells). Note the expression of p16 in cells selected in (i.e., grown in the presence of) adriamycin. The left most lane of FIG. 7B shows the level of signal from 200 ng/well of purified p16 protein as a marker protein.

FIG. 7C shows p16 expression in MCF7 cells, MCF7/AR cells (400 uM adriamycin resistant), MCF7/VLB1, MCF7/VLB10 nM cells (resistant to 10 nM vinblastine), MCF7/VCR2 cells (resistant to 2 nM vincristine), MCF7/VCR20 cells (resistant to 20 nM vincristine), MCF7/Mito78 nM cells (resistant to 78 nM mitoxantrone), three different extracts of white blood cells, CEM cells (lymphoid T-cell leukemia), SKOV3 cells (ovarian cancer cells), and total cell extract from normal mammary gland. Note p16 expression is present only in adriamycin resistant MCF7/AR breast cells. The left most lane of FIG. 7C shows the level of signal from 200 ng/well of purified p16 protein as a marker protein.

FIG. 7D shows the p16 expression in CEM cells, CEM/VLB 0.1 μM cells (resistant to 0.1 μM vinblastine), CEM/VLB 1 μM cells (resistant to 0.1 μM vinblastine), CEM/AR 0.8 μM cells (resistant to 0.8 μM adriamycin), CEM/AR 10 μM cells, (resistant to 10 μM adriamycin), MOTL4 cells, MOTL4/VLB 25 nM cells (resistant to 25 nM vinblastine), MOLT4/AR 250 nM cells (resistant to 250 nM adriamycin), MOLT4/AR 500 nM cells (resistant to 500 nM adriamycin), and K562 cells. MCF7/AR cells (adriamycin resistant) were used as a positive control. FIG. 7E shows p16 expression, for comparison purpose, in MCF7 and MCF7/AR cells versus that in MDA-MB-231 and MDA-MB-231/AR cells; and versus that in H69 and H69/AR cells. In addition, FIG. 7E shows p16 expression in OVCAR3 ovarian cancer cells and extracts from normal human ovary, prostate, brain and lung tissues. Note the level of signal from 200 ng/well of purified p16 protein as a marker protein in the left-most lane of FIG. 7E.

In addition, a third test, namely the p16 mRNA overexpression RT-PCR assay (p16 mRL test) was performed to determine p16 mRNA levels per cell. For this test, the following methods were used:

Primers used:

| | |
|---|---|
| Ap57 (p16-Forward): | |
| 5′ ATACGCGGATCCACCATGGAGCCTTCGGCTGACT | |
| GG 3′ | |
| Ap58 (p16-reverse): | (SEQ ID NO:_) |
| 5′ AAATTTAAAGCGGCCGCTCAGCTAGCGTAATCTG | (SEQ ID NO:_) |
| GTACGTCGTATGGGTAATCGGGGATGTCTGAGGG 3′ | |

Total RNA was extracted from approximately 1-5×10⁶ cells using the RNeasy Mini extraction kit (commercially available from Qiagen Inc., Valencia, Calif.) following the manufacturer's instructions. Approximately 1-2 ug of the resulting RNA was used for each RT-PCR reaction. RT-PCR was performed using the Ready-To-Go RT-PCR beads commercially available from Amersham Biosciences (Piscataway, N.J.) following the manufacturer's instructions. Briefly, the beads were resuspended in RNase-free water and 15 pmoles of each of the p16 specific primers were added together with 0.5 mg of random pdN6 primer (Amersham Biosciences) and 1-2 ug of the template RNA. Thermal cycling was performed using a Perkin-Elmer 9600 PCR instrument using the following parameters: 42° C. for 30 min. followed by denaturation at 95° C. for 5 min., and 35 cycles of denaturation at 95° C. for 30 sec., annealing at 55° C. for 30 sec. and polymerization at 72° C. for 1 min. RT-PCR products were analyzed by loading 10 ml on a 1% agarose gel stained with Ethidium Bromide. Visualization of bands was done under a UV-illuminator and images were analyzed using the Alpha Imaging Software (Alpha Innotech Corp., San Leandro, Calif.).

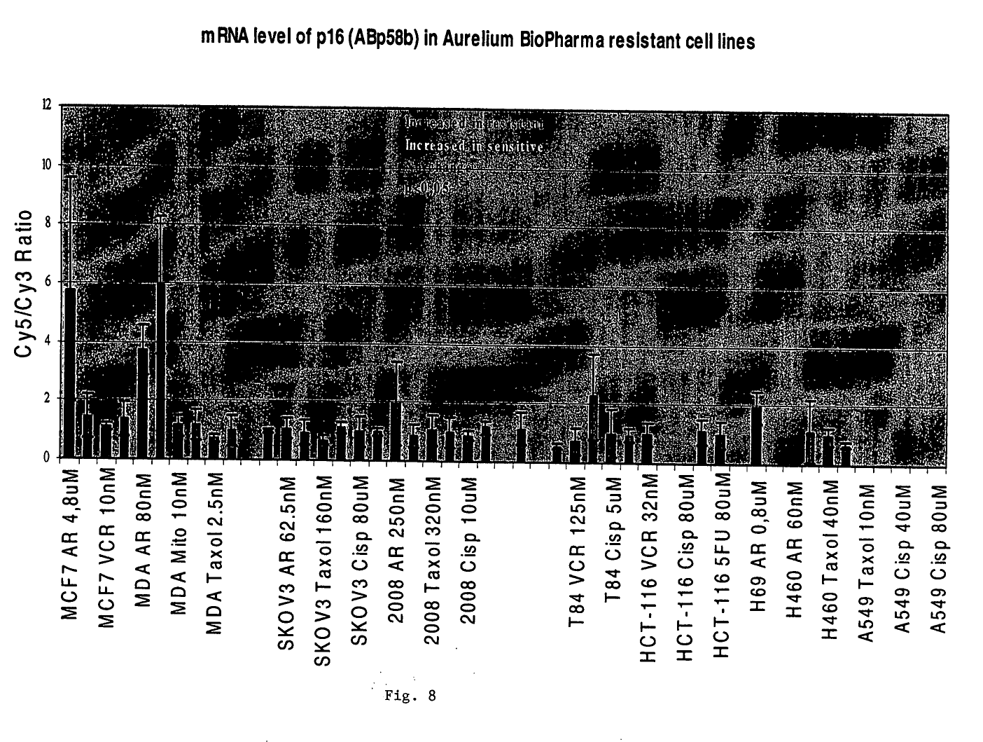

As shown in FIG. 8, cells that were resistant to adriamycin showed an increase in p16 mRNA as compared to their adriamycin-sensitive parents The Cy5/Cy3 ratio in the parent p16 is set at 1. Cy5 labels the mRNA of the adriamycyin-resistant cells and Cy3 labels the mRNA of the adriamycyin-sensitive cells.

The levels of p16 protein and mRNA, as determined with the SITp16 and p16 mRL assays, scored zero (undetectable) in a variety of control normal sample tissues tested. These control normal tissues included white blood cells obtained from 3 normal individuals (FIG. 7C), as well as normal human tissue protein lysates (obtained from Clonetech) of ovary, prostate, brain, and lung tissue (FIG. 7E) and breast mammary gland (FIG. 7C). As shown in Table II, FIGS. 7A-7E, and FIG. 8, the levels of p16 protein and mRNA expressed by a number of solid tumor cell lines correlated well with their degree of ADR drug resistance.

MCF7 and MCF7/AR were used as negative and positive controls, respectively, in the SITp16, SART, and p16 mRL tests. Comparison with data for p-glycoprotein, revealed that the levels of p-glycoprotein and mRNA in these cell lines were not proportional to their level of ADR resistance (see FIG. 4).

In another example, MDA-MB-231 is a human breast tumor cell line that is sensitive to adriamycin and expresses negligible amounts of p16 protein and mRNA (FIGS. 7B and 8).

Another third example is H69, a human lung cell line that is resistant to ADR.

In some instances, as the panel of cell lines tested was limited, some questions remain. For example, the ovarian tumor cell lines in the SKOV3 series did not express p16 protein (FIG. 7B), however, no ADR-resistant SKOV3 cell line was available for analysis. In contrast, OVCAR3, another ovarian cell line that was ADR-resistant, expressed p16 protein at very high levels (see FIG. 7E).

The correlation between the level of ADR drug resistance and the level of expression of the p16 gene was specific for ADR drug resistance, as the same cell lines that were selected for drug resistance to other types of drugs (e.g., mitoxantrone, taxol, vincristine or vinblastin) without multidrug resistance to ADR, did not express elevated levels of p16 protein. For example, no increase in p16 expression was observed in the melphalan resistant human prostate cancer PC3/Mel cells, as compared to its melphalan-sensitive parent PC3 cells (FIG. 7A, compare lanes 5 and 6).

MCF7NLB 1 nM, MCF7/VLB 10 nM, MCF7/Mito 78 nM, and MCF7/CisP are cell lines that were resistant to the indicated concentrations of drug, and were not MDR to ADR, nor did they express p16 protein (FIG. 7C shows p16 expression). The same situation is illustrated in the MDA-MB-231 cell line series shown in FIG. 7B, lanes 1-3) where derivative cell lines resistant to various concentrations of mitoxantrone were not also MDR to ADR, and did not express p16 protein.

Comparison between the expression levels of the genes known to be responsible for drug resistance in the above cell lines indicated that the expression of p-glycoprotein genes, vault protein gene, glutathione transferase gene, etc, were not proportional to the expression of p16 gene (data not shown). The results suggest that p16 gene does not regulate the expression of the p-glycoprotein gene, vault protein gene, glutathione transferase gene, etc.

Although the correlation between ADR resistance level and p16 protein expression level held true for the solid tumor cell lines tested, the hematological tumor cell lines all tested negative for p16 protein and mRNA expression. Table II shows that the human acute promyelocitic leukemia cell line HL60 and its ADR resistant derivative cell line HL60/AR, both do not express p16 protein. FIG. 7D likewise shows that no p16 expression was observed in adriamycin-resistant MOLT4 leukemia cells (compare lanes 6, 8, and 9), indicating that the correlation of increased resistance to adriamycin and increased p16 expression is not true in human leukemia cells. Another hematological cell line series tested, CEM, an acute lymphoblastic leukemia, gave similar results (see Table II and FIG. 7D). All the hematological tumor cell lines that were tested were found to express no p16 protein or mRNA by SITp16 or p16 mRL analysis, regardless of whether they were resistant or sensitive to adriamycin. This includes some cells, such as CEM/AR 0.8 uM and CEM/ AR 10 uM, MOTL4/AR 250 nM and MOTL4/AR 500 nM, that were very highly resistant to adriamycin (ADR).

The fact that p16 is not expressed in the above hematological tumor cell lines would confirm previous reports (Taniguchi et al., Leukemia 13: 1760-1769, 1999) that the p16 gene is silenced in these cell lines either through deletion, point mutation, or methylation, and cannot be expressed. Thus although the SrTp16 test is useful for predicting the level of ADR resistance in solid tumor cell lines, it does not appear to be useful for predicting ADR drug resistance levels in hematological tumor cell lines. Analysis of p16 expression in fresh hematological tumor samples would help us to determine whether this is generally the case for hematological tumors or whether the silencing of the p16 gene occurs in the hematological tumor cell lines because of tissue culture selection, as has been reported by others (Taniguchi et al., Leukemia 13: 1760-1769, 1999).

Schematic FIG. 2, as described above, illustrates how the SITp16 test and/or additional tests based on the RT-PCR quantification of p16 mRNA (or other methods of detecting p16 protein levels, such as 2D gels) could be used as a predictive test in the clinic to determine the level of ADR resistance in a clinical solid tumor specimen. The test could be further miniaturized and speeded up with the use of dot blotting technologies and fluorescent probes (anti-p16 antibody or primers).

Finally, in a modification of the SITp16, an experiment was performed to determine if p16 expression was induced following a short-term exposure of adriamycin-sensitive cells to adriamycin. For these studies, MCF7 cells were treated 3 days with the EC50 amount of adriamycin (i.e., the effective concentration median or middle of a dose response curve -for MCF7 cells and adriamycin, this is 40 nM. (Note that EC50 is the effective concentration median or middle of a dose response curve, while IC₅₀=inhibitory concentrations by 50%. Total cell lysates (i.e., extracts) were then prepared from these adriamycin-treated cells (called MCF7 EC50 Doxo), as well as from an equal number of MCF7 cells (drug sensitive), MCF7/AR (adriamycin-resistant cells), and OVCAR3 cells (an ovarian cancer cell line from a patient who was treated with adriamycin). The lysates were resolved by SDS-PAGE, transferred to a nitrocellulose membrane, and probed with p16^{INK4a} specific monoclonal antibody (α-p16^{INK4a} (Ab-4, Neomarkers clone 16P04, JC2 commercially available from Lab Vision, Freemont, Calif.).

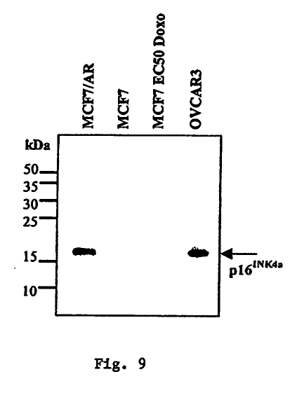

Studies were next performed to determine if a short duration of exposure to adriamycin would cause upregulation of p16 expression in MCF7 cells. For this study, MCF7 cells were cultured in the presence of Doxorubicin (or adriamycin) for 3 days at a concentration, 50 nM adriamycin, that was known to kill 50% of MCF7 drug-sensitive cells. After the 36 hours of culture, the remaining live cells were isolated and cell lysates prepared to determine if doxorubicin or adriamycin treatment of cells transiently induced p16 expression. Interestingly, as shown in FIG. 9, the 36 hour treatment of MCF7 cells with 50 nM adriamycin did not result in these cells' upregulation of p16 expression. (Note that MCF7/AR cells serve as a positive control for p16 expression.) Thus, although treatment of the MCF7 EC50 Doxo will eventually result in adriamycin resistant, p16 expressing cells, this short duration of contact with adriamycin was insufficient to induce p16 expression in these cells.

### EXAMPLE 3

### Inhibition of p16 Results in Increased Adriamycin Sensitivity in Tumor Cells

Further experiments were performed to inhibit p16 expression using RNA interference, and determine if cells having reduced p16 expression also had increased sensitivity to adriamycin.

For these experiments, the following methods were used:

I. p16 siRNA transfections

The genomic sequence of p16INK4a is provided in SEQ ID NO: 2 (and GenBank Accession Nos. NM 000077.2 GI: 17738299. Two siRNA duplexes were chosen that targeted unique sequences in the p16 INK4a locus: CAACGCACCGAATAGTTAC (p16 siRNA Duplex I; SEQ ID NO: ______) and CGGAAGGTCCCTCAGACAT (p16 siRNA Duplex II; SEQ ID NO: ______). The first sequence is from Exon 1a, spanning nucleotides 385-404 counting from the start codon. The second sequence is from Exon 3, spanning nucleotides 714-733 of the p16 mRNA. Both sequences are specific for the p16INK4a transcript and are not in regions homologous to the other INK4a inhibitors, and do not overlap P¹⁹ARF sequences.

To target p16 siRNA Duplex I, the following oligonucleotide was made:

| | |
|---|---|
| Sense Sequence: | 5′ CAACGCACCGAAUAGUUACtt 3′ |
| Antisense Sequence: | 5′ GUAACUAUUCGGUGCGUUGtt 3′ |

To target p16 siRNA Duplex II, the following oligonucleotide was made:

| | |
|---|---|
| Sense Sequence: | 5′ CGGAAGGUCCCUCAGACAUtt 3′ |
| Antisense Sequence: | 5′ AUGUCUGAGGGACCUUCCGtt 3′ |

In addition, a mutated p16^{INK4a} sequence was targeted with siRNA. These siRNA had the following sequences (where the mutated residues are underlined):

| | |
|---|---|
| Sense Sequence: | 5′ CAACGCACCUAAUAGUUACtt 3′ |
| Antisense Sequence: | 5′ GUAACUAUUAGGUGCGUUGtt 3′ |

For an siRNA transfection, 1 nmole of the annealed siRNA duplex (the siRNA were provided annealed from the manufacturer Dharmacon Inc., Lafayette, Colo.) was mixed in a tube with 1.4 mL of Opti-MEM reagent (commercially available from InVitrogen Corp., Carlsbad, Calif.). In another tube, 85 μL of Oligofectamine reagent (commercially available from InVitrogen) was mixed with 600 μL of Opti-MEM. The two solutions were combined, mixed gently by inversion and incubated for 20 minutes at room temperature (i.e., 25° C.). The resulting solution was added to the cultured cells drop-wise in a 10 cm dish having approximately 40-50% confluent cells.

Cells were also transfected with two control siRNAs which targeted the GFP (green fluorescent protein, which is not naturally expressed by these cells and serves as a negative control) and bcl-2 genes (which is naturally expressed by these cells and serves as a positive control). The sequences for these siRNAs are as follows.

GFP siRNA duplex:

| | |
|---|---|
| Sense sequence: | 5′ PGGCUACGUCCAGGAGCGCACC 3′ |
| Antisense sequence: | 5′ PUGCGCUCCUGGACGUAGCCUU 3′ |

Bcl-2 siRNA duplex:

| | |
|---|---|
| Sense sequence: | 5′ GCUGCACCUGACGCCCUUCtt 3′ |
| Antisense sequence: | 5′ GAAAGGGCGUCAGGUGCAGCtt 3′ |

The expression levels of p16 protein and mRNA were quantified using the SITp16 and mRLp16 assays as described above. The SITp16 immunoblots were stripped and reblotted with control anti-actin and anti-bcl-2 antibodies as described above, in order to standardize for total protein levels in each sample.

II. Clonogenic Assays

a) Methylene Blue Staining. HeLa cells were transfected with the siRNA duplexes as described above. The following day the cells were harvested by trypsinization and 2×10⁴ cells/well were seeded in triplicate in 24-well plates. The cells were incubated for 4 days and stained with 0.5% Methylene Blue solution for 15 minutes at room temperature, washed, drained, and dried. The blue colonies were solubilized in 0.1% (v/v) SDS/PBS and the absorbance of the solution was determined by spectroscopy at 610 nm. The results are expressed as the average absorbance of triplicate wells.

b) CyQuant Cell Proliferation Assay This assay was used to measure cell proliferation based on DNA content. MCF7/AR cells or HeLa cells were transfected as described above and the next day seeded in a 96-well plate at 3×10³ cells/well. The plate was incubated for 48 hours at 37° C. The media was removed and 100 μL of CyQUANT GR dye cell lysis buffer (Molecular Probes) was added per well. The plates were incubated for 5 minutes at room temperature. The resulting fluorescence was measured in a microplate reader (WALLAC VICTOR-1420, commercially available from PerkinElmer, Inc., Boston, Mass.) using a 535 nm filter. Results were expressed as the average of quadruplicate cultures and were plotted with MS Excel. The number of cells was determined by extrapolation from a standard curve.

III. MTT Cytotoxicity Assays. 24 hours post-transfection, siRNA transfected cells were seeded into 96-well plates at 3×10³ cells/well. The cells were incubated for 16-24 hours. A range of concentrations of the indicated cytotoxic drugs was added and incubation was continued for an additional 48 hours. 20 μL of MTT (10 mg/mL) was added to each well and the plates were further incubated at 37° C. for 4 hours. The dye was solubilized with DMSO and absorbance measured at 570 nm using a multiwell plate reader (WALLAC). The results were expressed as the average of duplicate wells using the plate reader's Prism software (GraphPad).

In order to study the effect of inhibiting p16 protein expression, the two siRNA oligonucleotide duplexes were created as described above (see also Bond et al., Exp Cell Res. 292:151-156, 2004). These siRNAs were specific for the p16 mRNA and did not contain sequences that bound to any of the other INK4A family inhibitor mRNAs or to P19_{ARF}.

Two different ADR-resistant tumor cell lines (HeLa, ovary; MCF7/AR, breast) were transfected with the two different siRNA duplexes, and p16 gene expression levels were evaluated using the SITp16 test, immunoblotting with the p16^{INK4a}-specific monoclonal antibody, α-p16INK4a (Ab-4, clone 16P04, JC2; Neomarkers).

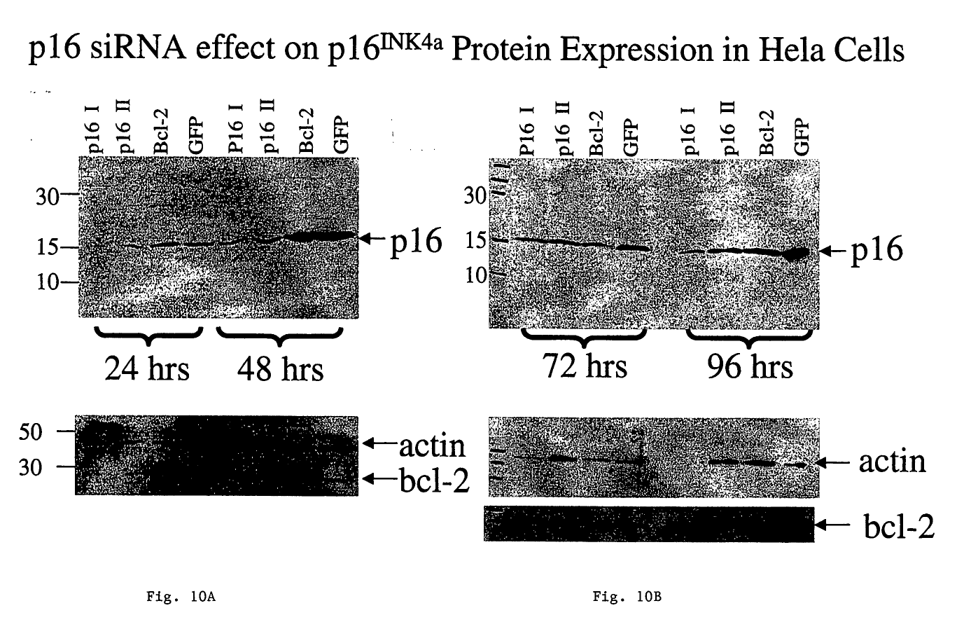

Expression of p16^{INK4a} protein in HeLa cells 24 hours and 48 hours (FIG. 10A) or 72 and 96 hours (FIG. 10B) following transfection with either p16 I siRNA or p16 II siRNA resulted in the reduced expression of p16 protein. As Table II below shows, the diminution was most dramatic 24 hours post-transfection

TABLE II
Percentage reduction in P16 protein expression
as compared to GFP siRNA transfected cells

| | |
|---|---|
| Hela cells | siRNA used |

| Hours post-transfection | siRNA P16 I | siRNA P16 II |
|---|---|---|
| 24 hrs | 90% | 88% |
| 48 hrs | 82% | 70% |
| 72 hrs | 62% | 58% |
| 96 hrs | 66% | 62% |

Controls included treatment of HeLa cells with bcl-2 and GFP siRNAs, and immunoblotting with specific anti-bcl-2 (NeoMarkers, Ab-1, Clone 100/05) and anti-actin monoclonal antibodies (pan-actin Ab-5, NeoMarkers, Clone ACTN05) (lower panel of FIG. 10A for 24 and 48 hours post-transfection; lower panels of FIG. 10B for 72 and 96 hours post-transfection).

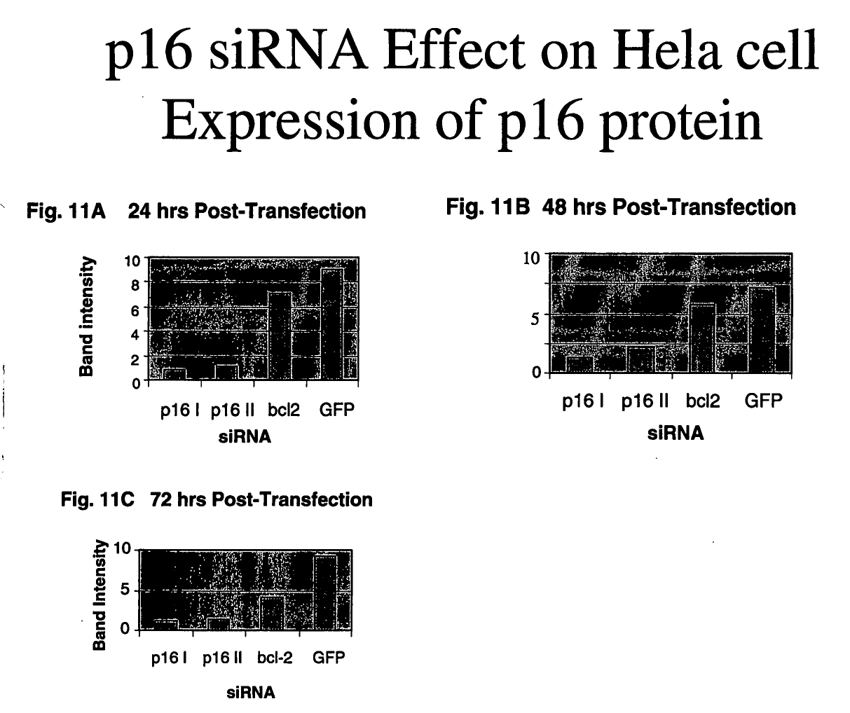

The knock down of p16 expression is more readily visualized in the quantitation of ban density from Western blotting analyses (using Image Master 2D software (Amersham-Pharmacia)). HeLa cell expression of p16^{INK4a} (with two different p16 siRNAs), bcl-2, and GFP proteins after 24 hours post-transfection (FIG. 11A), 48 hours post-transfection (FIG. 11B), and 72 hours post-transfection (FIG. 11C) is clearly reduced by transfection with p16 I siRNA or p16 II siRNA. Both of the p16 siRNAs selectively inhibited the expression of p16^{INK4a} protein at 24, 48, or 96 hours post-transfection as quantified by Image Master 2D software (Amersham-Pharmacia).

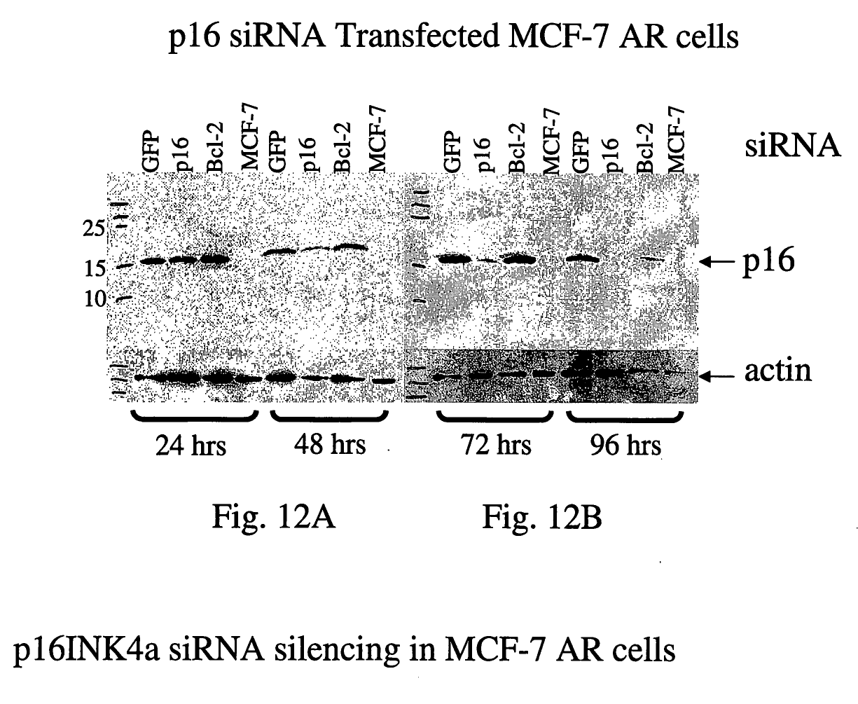

Clear inhibition of p16 protein and mRNA levels was also observed in the MCF7/AR p16 siRNA transfectants at 48 hours post-transfection (80% decrease in p16 protein level relative to cells transfected with GFP siRNA), but not in the control Bcl-2 and GFP siRNA transfected cells (see FIG. 12A). Inhibition was also observed at 72 hours (94% decrease in p16 protein level relative to cells transfected with GFP siRNA), and 96 hours after transfection (99% decrease in p16 protein level relative to cells transfected with GFP siRNA) (see FIG. 12B). Without stripping (which may remove some intensity in signal), the SITp16 blots were reblotted with two control monoclonal antibodies, anti-actin and anti-bcl-2 (see lower panels of FIGS. 12A and 12B, respectively. Note that actin is the control for gel loading, although the BCL2 band can be seen as the fainter band below actin to show the relative level of total protein loaded onto each gel well.

The relative effect of siRNA treatment on reversing ADR sensitivity of MCF7/AR cells and on p16 gene expression was assessed using the SITp16 and SART assays in the presence or absence of ADR drug treatment of the cells. As can be seen results in FIGS. 12A and 12B, p16 siRNA knocked down the levels of p16 protein, and reversed ADR drug sensitivity concomitantly. These results show that the drug resistance reversal effects of siRNA can overcome p-glycoprotein mediated ADR resistance in MCF7/AR cells as well as ADR resistance in HeLa cells.

Thus p16 gene expression appears to control the expression of Topoisomerase II alpha, p53 Cathepsin B, and Stratifin genes, which may be involved in MDR/drug resistance.

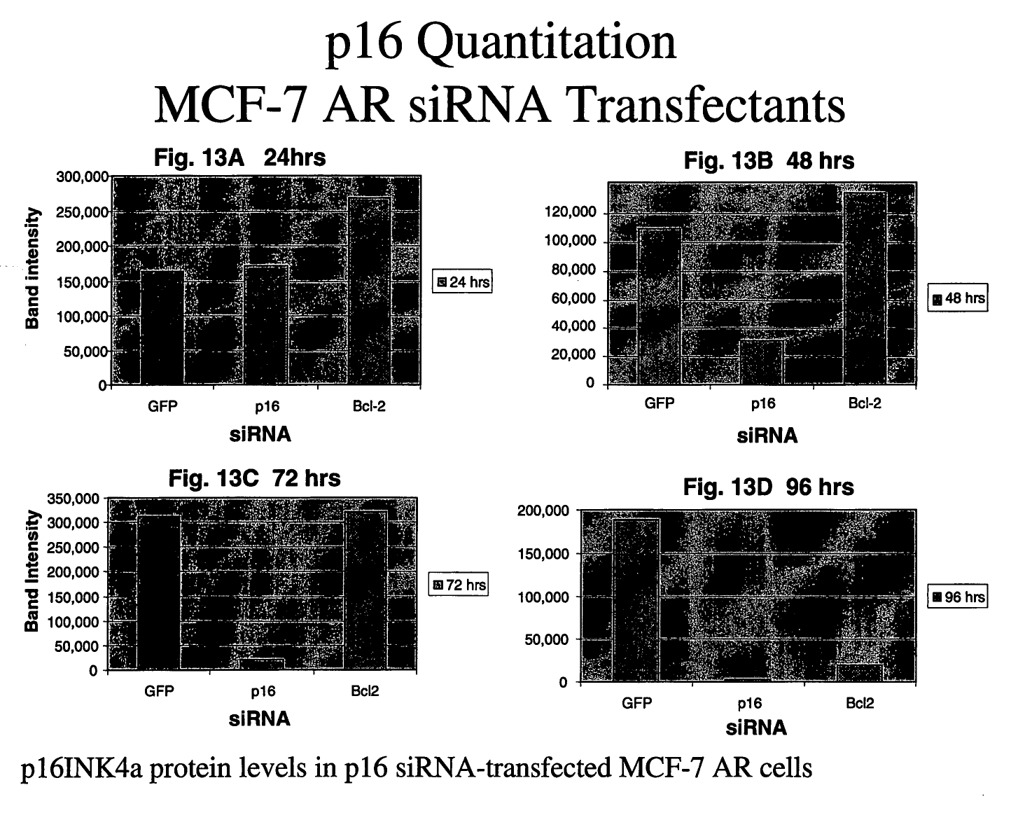

FIGS. 13A-13D present the results of the data analysis from FIGS. 12A and 12B, as quantified by Image Master 2D software (Amersham-Pharmacia): the levels of p16 protein in MCF-7 AR cells were reduced by siRNA p16 I transfection approximately 10 fold and by siRNA p16 II transfection approximately 8.4 fold relative to the control bcl-2 and GFP siRNA-transfected cells at 72 hours.

Estimates of p16 proteins levels following transfections were determined by densitometric analysis using 2D Image Master Analysis software from Amersham-Pharmacia Biotech.). Table II shows relative decrease in p16 protein (as % of GFP control transfection) over time in Hela cells.

A CyQuant cell proliferation assay was next performed on cells grown in the absence of adriamycin to measure the effect of transfection with 100 nM of p16 siRNAs on HeLa and MCF7/AR cell proliferation. As shown in FIGS. 14A-14B, p16 siRNA I, but not control bcl-2 or GFP siRNAs, were found to markedly stimulate cell proliferation in both HeLa cells (FIG. 14A) and MCF-7/AR cells (FIG. 14B) by approximately 2.5 fold-3 fold and simultaneously reversed ADR resistance, as measured with the CyQuant cell proliferation assay. The rate of cell proliferation in each set of cell lines was inversely proportional to the level of p16 gene expression, with the highest rates of proliferation observed in maximally p16 siRNA-inhibited cell lines and the slowest rates of proliferation observed in untreated cells with the highest p16 gene expression (FIG. 14). These results suggest that highly ADR-resistant cell lines are very similar to senescent normal cells in terms of having zero proliferation and very high p16 gene expression levels (Bond et al., supra). Conversely, senescent cells in general may be more highly ADR-resistant than non-senescent cycling cells, thus the strategy of designing ADR-reversal agents that push cells towards senescence may, surprisingly, actually increase ADR and/or MDR-resistance rather than decreasing drug-resistance.

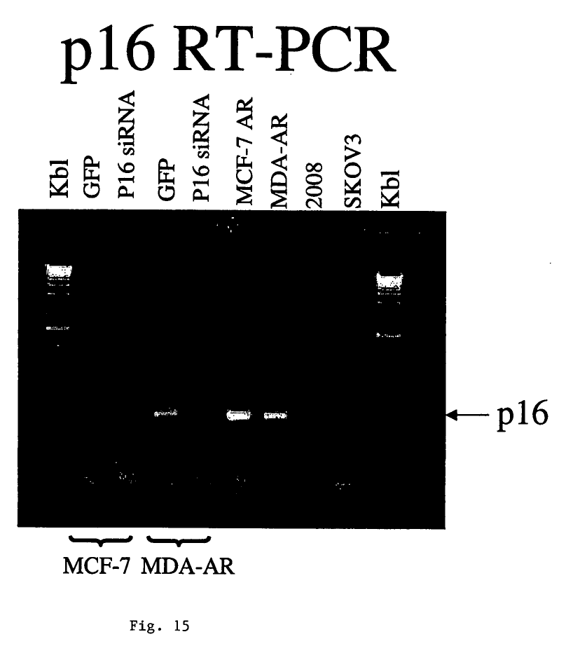

The p16 mRL test was next used to confirm that p16 siRNA also had an effect on the amount of p16 mRNA. For these studies, the p16 mRL test, which is described above in Example 3, was employed on MCF-7 or MDA-AR cells that had been transfected 4 days previously with GFP siRNA or p16 siRNA, as described above. For comparison purposes, the level of p16 mRNA in untransfected MCF-7 AR, MDA-AR, 2008, and SKOV3 cells was also assessed. The p16 primers amplify an approximately 477 base pair p16 cDNA fragment. As shown in FIG. 15, p16 mRNA was present in relatively high quantities in MDA-AR cells transfected with gfp siRNA. In contrast, MDA-AR cells transfected with p16 siRNA I had lower amounts of 16 mRNA. Untransfected MCF-7 AR and MDA-AR cells, being adriamycin-resistant, also, not unexpectedly, expressed p16 mRNA. P16 mRNA was also found in small amounts in 2008 cells.

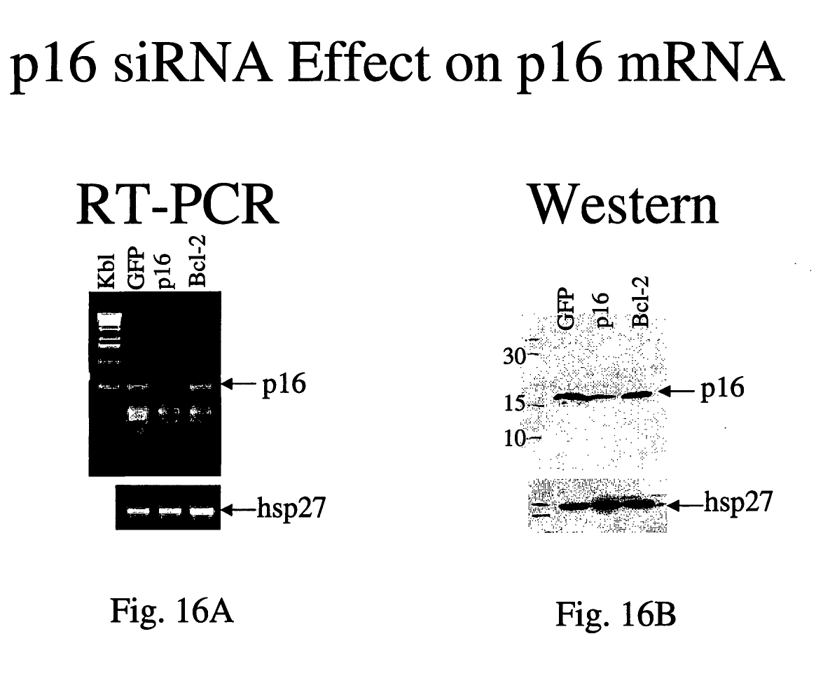

The reduction in p16 mRNA by p16 siRNA parallels the reduction in p16 protein by p16 siRNA. As shown in FIG. 16A, the amount of p16 mRNA was noticeably reduced in Hela cells transfected with p16 siRNA as compared to cells transfected with GFP siRNA or bcl-2 siRNA, as determined by the p16 mRL test. Equal amounts of mRNA in all lanes was confirmed by equivalent amounts of hsp27 mRNA (see bottom of FIG. 16A). This reduction in p16 mRNA correlated well with a reduction in p16 protein (FIG. 16B), as determined by the SITp16 test. Again, equal loading of all lanes was confirmed by the presence of hsp27 protein (see bottom of FIG. 16B).

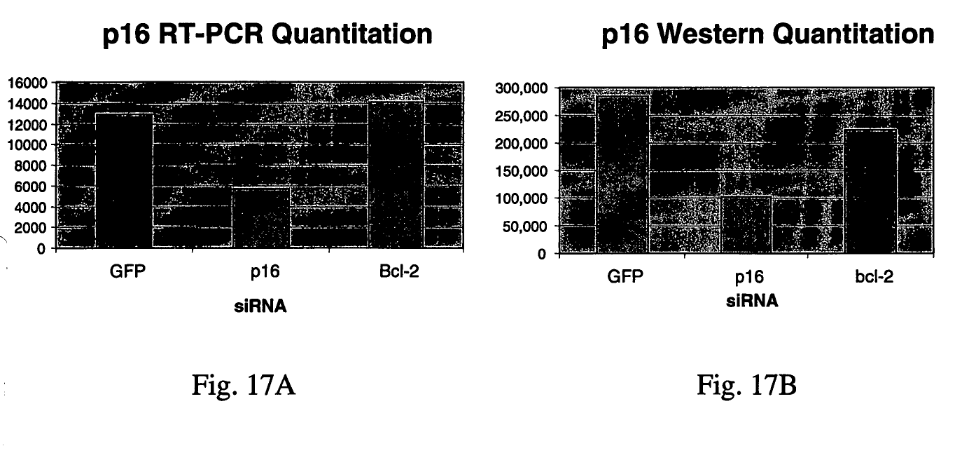

The band densities of the blots shown in FIGS. 16A (Program Alpha Imager) and 16B (Program 2-D Image Master) were quantitated using densitometric analysis. As shown in FIG. 17A, the amount of p16 mRNA in cells transfected with p16 siRNA was reduced by 56% as compared to cells transfected with GFP siRNA or bcl-2 siRNA. Similarly, the amount of p16 protein in cells transfected with p16 siRNA was reduced by 64% as compared to cells transfected with GFP siRNA or bcl-2 siRNA (FIG. 17B).

Experiments were next done comparing the ability of the mutated p16^{INK4a} siRNA described above (p16mut) with wild-type p16 siRNA. For these studies, MDA/AR cells were transfected with gfpsiRNA (control), p16 siRNA I, or p16 mut siRNA. P16 protein levels were assessed 48 hours post-transfection using the SITp16 test. As shown in FIG. 18, cells transfected with p16siRNA, but not p16 mut siRNA, showed reduced p16 protein levels. As compared to the amount of p16 protein present in cells transfected with gfp siRNA, the cells transfected with p16 siRNA showed a 79% reduction in p16 protein expression. Equal loading of all lanes was confirmed by the equivalent presence of ANX-I in all lanes (see bottom of FIG. 18A). This experiment was repeated for both 2 days and 4 days post-transfection. As shown in FIG. 18B, MDA/AR cells transfected 2 or 4 days previously with p16 siRNA showed 92% and 87% reductions in p16 protein levels, respectively, as compared to cells transfected with p16 mut siRNA or gfp siRNA. Again, equal loading of all lanes was confirmed by the equivalent presence of ANX-I in all lanes (see bottom of FIG. 18B).

Similar results were observed for MCF-7/AR cells. For these studies, the cells were transfected, as described above, with either gfp siRNA, p16 mut siRNA, or p16 RNA. Two days later, the level of p16 protein in the transfected cells was assessed using the SITp16 test. As can be seen from FIG. 19A, a 60% decrease in p16 protein levels was observed in cells transfected 48 hours earlier with p16 siRNA I as compared to cells transfected 48 hours earlier with either gfp siRNA or p16mut siRNA. This reduction in p16 expression was maintained in cell transfected with p16 siRNA even after the cells had been transfected 4 days earlier (see FIG. 19B).

### EXAMPLE 4

### p16 Inhibition Results in Increased Sensitivity to Other Chemotherapeutic Agents in Tumor Cells

Additional experiments were performed to determine if inhibition p16 expression using RNA interference (resulting in cells having reduced p16 expression) also led to increased sensitivity to chemotherapeutic agents other than adriamycin.

For these studies, the methods described in Example 3 for transfecting HeLa and MCF7/AR cells with siRNAs to GFP, p16 siRNA I, or Bcl-2. Forty-eight hours following transfection, the cells were incubated in different concentrations of Cisplatinum, adriamycin (also called doxorubicin), taxol, melphalan, mitoxantrine, mitomycin C, thio tepa, vinblastine, vincristine, and chlorambucil for 48 hours. Following culture in these chemotherapeutic agents, the cells were subjected to a clonogenic assay, as described above in Example 3.

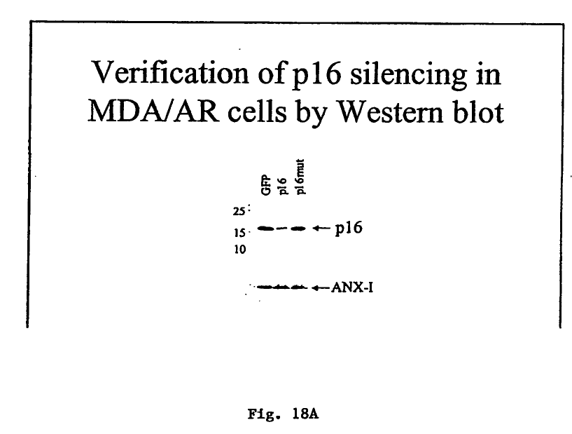

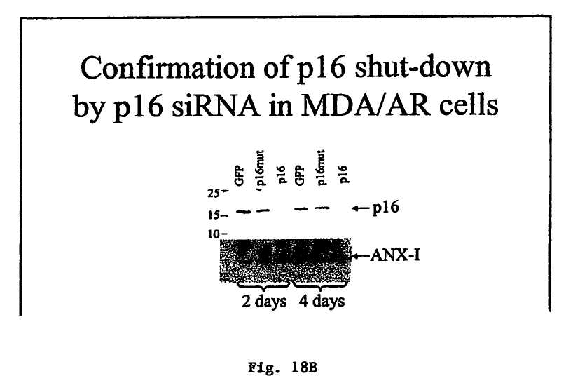

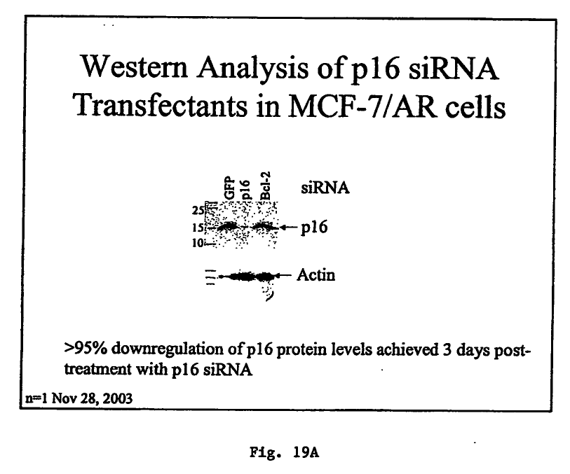

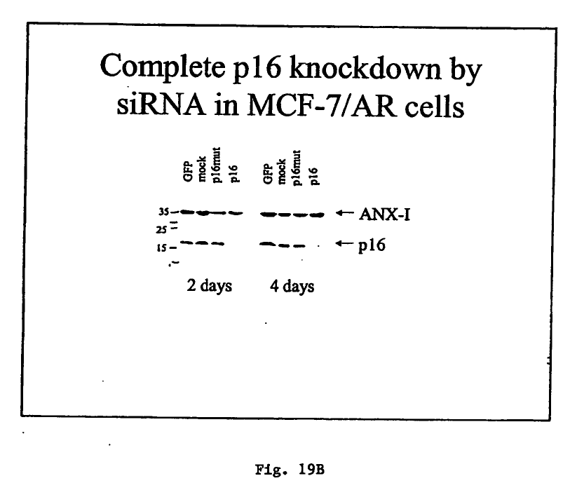

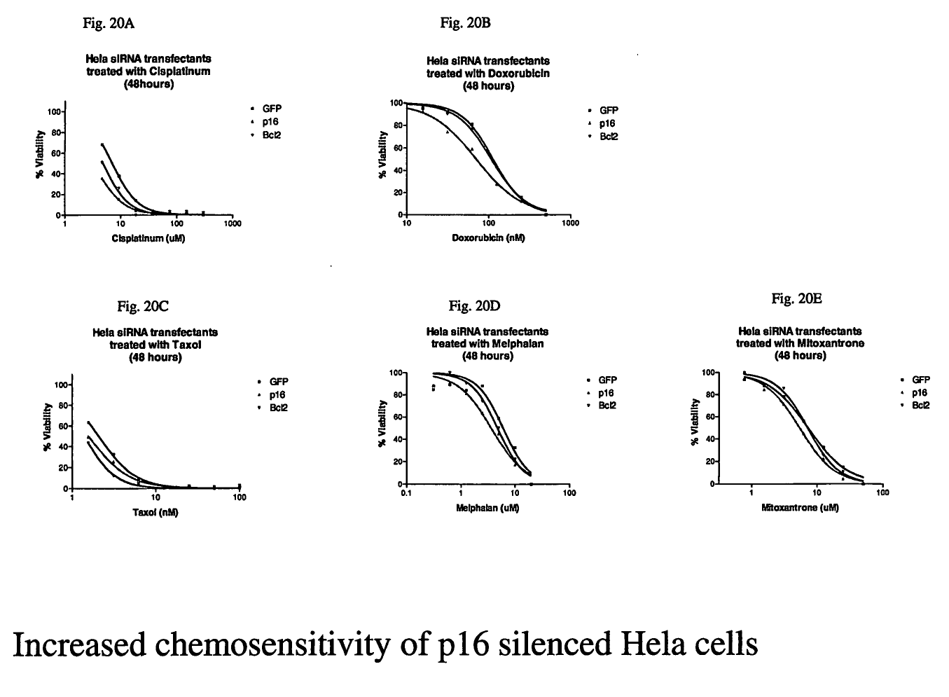

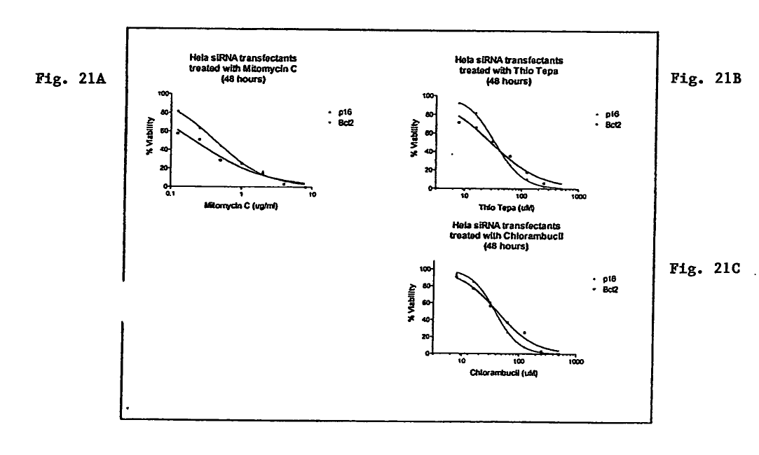

As can be seen in FIGS. 20A through FIG. 20E, HeLa cells transfected with p16 siRNA showed reduced viability in the presence of cisplatinum, adriamycin, melphalan, and mitoxantrone in comparison to those HeLa cells transfected with gfp siRNA or Bcl-2 siRNA. Further, when compared with HeLa cells transfected with Bcl-2 siRNA, HeLa cells transfected with p16 siRNA showed reduced viability in the presence of thio tepa and chlorambucil at high concentrations (see FIGS. 21A-21C).

Using the MTT cytotoxicity assay (as described in Example 3), the IC₅₀ (i.e., the concentration at which 50% of the cells are still viable) of HeLa cells transfected with p16 siRNA for various chemotherapeutic agents was next assessed. As Table III below shows, the IC₅₀ results for HeLa cells transfected with p16 siRNA I is markedly different than the IC₅₀ results for HeLa cells transfected with gfp siRNA.

TABLE III

| IC₅₀ Results (MTT) | GFP siRNA | P16 siRNA |
|---|---|---|
| Doxorubicin (nM) | 113.1 (R2 = 0.9955) | 70.39 (R2 = 0.9918) |
| ThioTepa (μM) | 28.73 (R2 = 0.9756) | 33.92 (R2 = 0.9981) |
| Mitoxantrone (μM) | 7.282 (R2 = 0.9942) | 5.411 (R2 = 0.9947) |
| Melphalan (μM) | 6.018 (R2 = 0.9368) | 3.63 (R2 = 0.9656) |
| Chlorambucil (μM) | 41.16 (R2 = 0.9878) | 37.99 (R2 = 0.9990) |
| Mitomycin C (μM) | 0.2135 (R2 = 0.9639) | 0.42209 (R2 = 0.9944) |

Note that R²=coefficient of determination or Regression of the curve. R² corresponds to the best fit of the curve, with reference to the MTT analysis. This value is derived using PRISM Software.

Thus, Table III shows that for HeLa cells, reduction of p16 expression resulted in the cells' increased sensitivity to adriamycin, mitoxantrone, melphalan, and chlorambucil as compared to HeLa cells transfected with gfp siRNA.

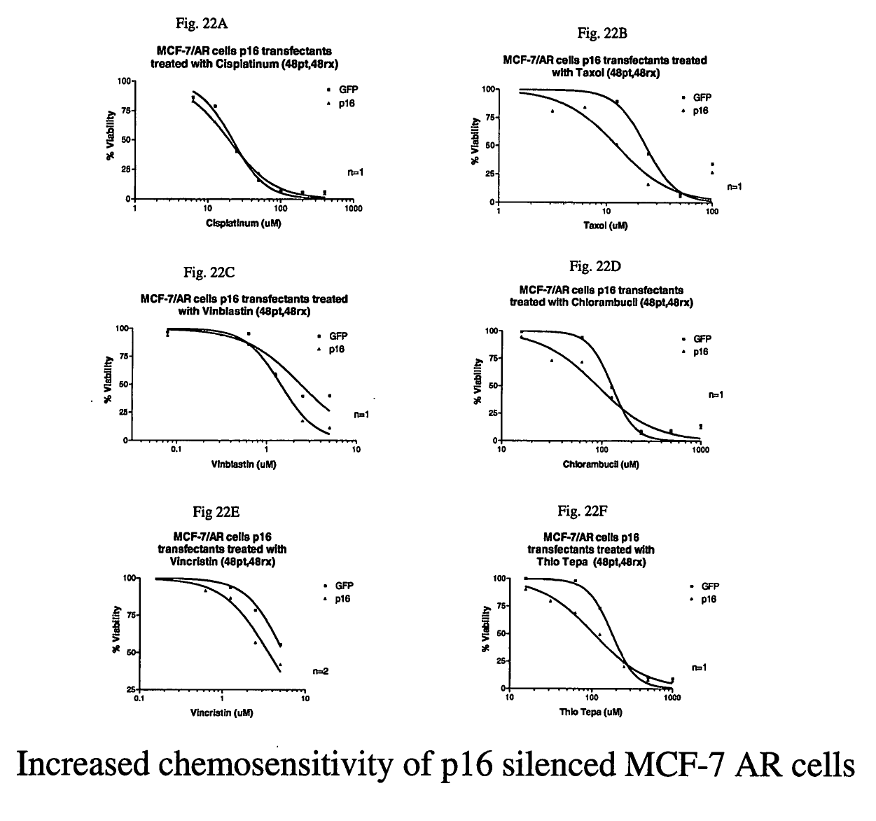

Similarly, MCF7/AR cells transfected with p16 siRNA (a) showed reduced viability in the presence of taxol and vincristin in comparison to those MCF7/AR cells transfected with gfp siRNA (see FIGS. 22B and 22E), (b) showed reduced viability in the presence of cisplatinum, chlorambucil, and thio tepa at low concentrations (see FIGS. 22A, 22D, and 22F), and (c) showed reduced viability to vinblastin at high concentrations (see FIG. 22C).

Using the MTT cytotoxicity assay, the IC₅₀ (i.e., the concentration at which 50% of the cells are still viable) of MCF7/AR cells transfected with p16 siRNA for various chemotherapeutic agents was next assessed. As Table IV below shows, the IC₅₀ results for MCF7/AR cells transfected with p16 siRNA I is markedly different than the IC₅₀ results for MCF7/AR cells transfected with gfp siRNA.

TABLE IV

| IC₅₀ Results (MTT) | GFP siRNA | P16 siRNA |
|---|---|---|
| Taxol (nM) | 23.58 (R2 = 0.8712) | 12.54 (R2 = 0.9025) |
| Cisplatinum(μM) | 21.77 (R2 = 0.9829) | 19.43 (R2 = 0.9966) |
| ThioTepa (μM) | 177.2 (R2 = 0.9863) | 105.7 (R2 = 0.9886) |
| Vinblastin (nM) | 2.376 (R2 = 0.8500) | 1.423 (R2 = 0.9869) |
| Chlorambucil (μM) | 125.3 (R2 = 0.9795) | 89.1 (R2 = 0.9574) |

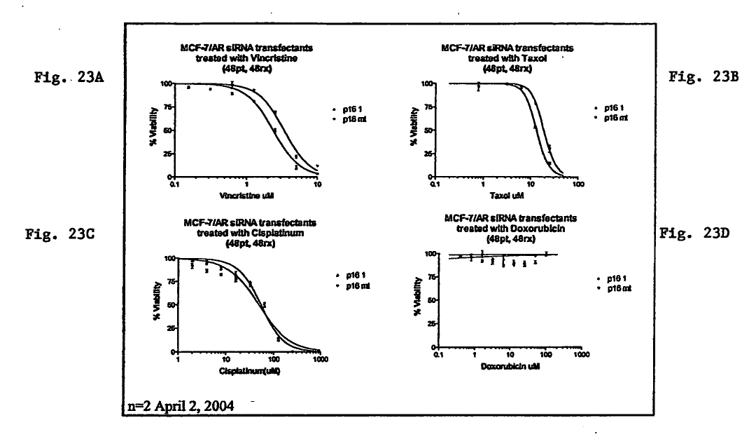

Interestingly, although the p16 mut siRNA was not able to reduce p16 protein expression when transfected into MCF7/AR cells (see, e.g., FIGS. 19A and 19B), the susceptibility of cells transfected with p16 mut siRNA to various chemotherapeutic agents, including adriamycin (i.e., doxorubicin) was only slightly higher at high concentrations of these agents than the susceptibility to these agents by MCF7/AR cells transfected with p16 I siRNA (see FIGS. 23A-23D). This seems to indicate that even the slight reduction in p16 expression caused by p16 mut siRNA rendered the cells more susceptible to killing by these chemotherapeutic agents.

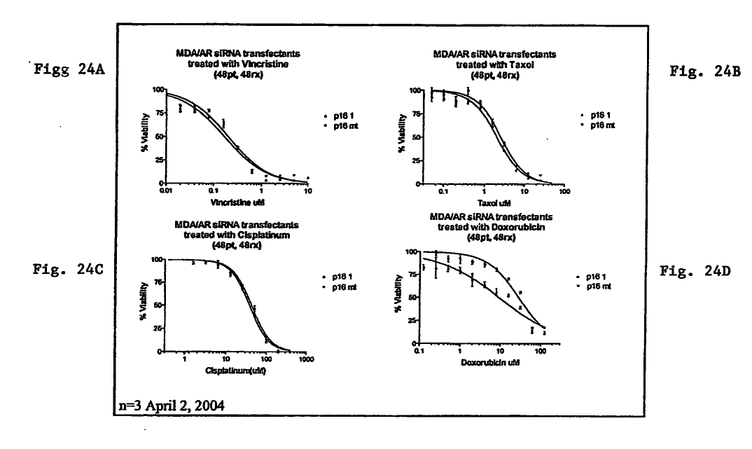

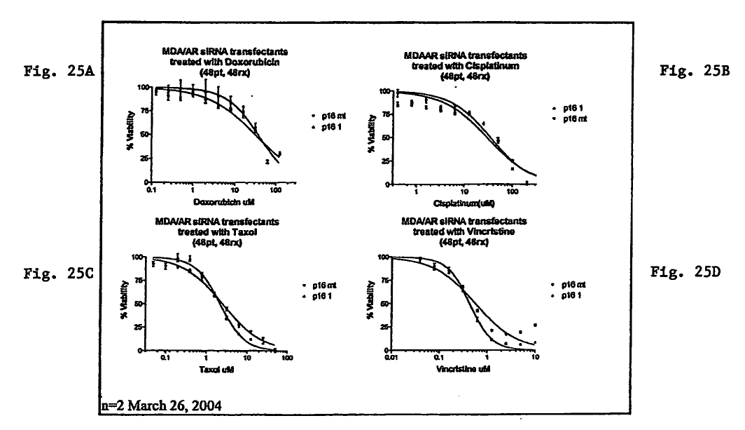

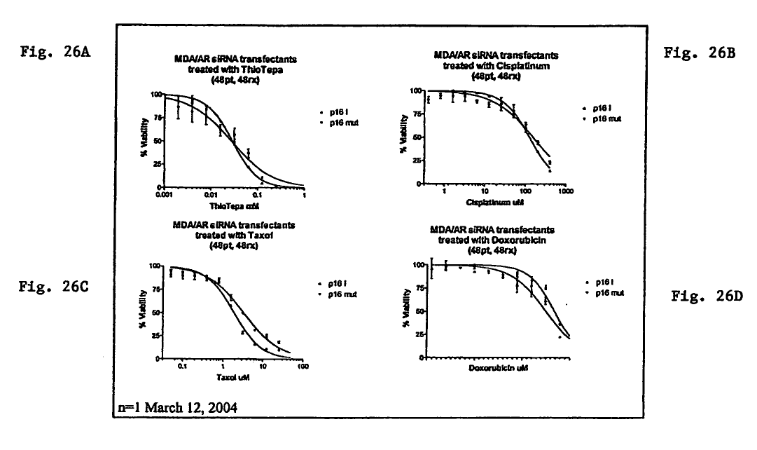

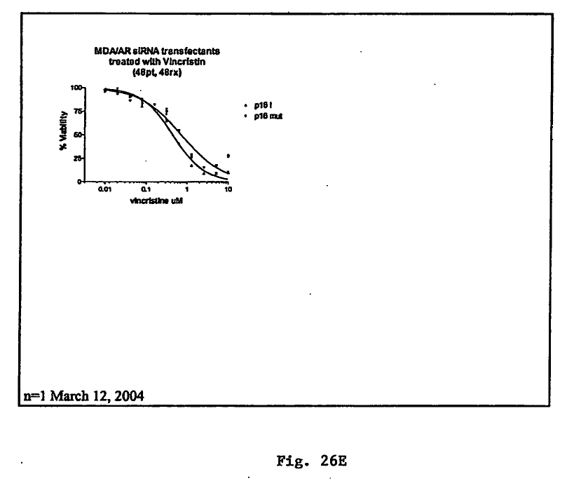

MDA/AR cells transfected with p16 siRNA showed reduced viability in the presence of high concentrations of adriamycin and taxol as compared to cells transfected with p16 mut siRNA (see FIGS. 24A-26E). For these studies, the cells were transfected 48 hours prior to culture for an additional 48 hours in the presence of the chemotherapeutic.

These results show that reducing p16 expression in adriamycin-resistant tumor cells results in the cells' increased sensitivity to a variety of chemotherapeutic agents.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

TABLE I

Tryptic peptide sequences of the protein
overexpressed in adriamycin-resistant human breast
tumor MCF7/AR cells, as determined by MALDI-TOF
MS.

| | | |
|---|---|---|
| Mass | Mass | Peptide sequence |

| | | | |
|---|---|---|---|
| PPeptides | submitted | matched | (Residues of SEQ ID NO:1) |

| | | | |
|---|---|---|---|
| 1 | 1398.803 | 1398.774 | ⁸⁸EGFLDTLVVLHR⁹⁹ |
| 2 | 1424.689 | 1424.681 | ⁴⁷RPIQVMMMGSAR⁵⁸ |
| 3 | 1713.940 | 1713.892 | ³⁰ALLEAGALPNAPNSYGR⁴⁶ |
| 4 | 1934.991 | 1933.988 | ¹⁰⁸DAWGRLPVDLAEELGHR¹²⁴ |
| 5 | 2220.975 | 2220.006 | ¹MEPAAGSSMEPSADWLATAAAR²² |
| 6 | 2220.975 | 2222.204 | ¹¹³LPVDLAEELGHERDARYLR¹³¹ |

| | |
|---|---|
| Ap57 (p16-Forward): | |
| 5′ ATACGCGGATCCACCATGGAGCCTTCGGCTGACT | |
| GG 3′ | |
| Ap58 (p16-reverse): | (SEQ ID NO:3) |
| 5′ AAATTTAAAGCGGCCGCTCAGCTAGCGTAATCTG | (SEQ ID NO:4) |
| GTACGTCGTATGGGTAATCGGGGATGTCTGAGGG 3′ | |

| | |
|---|---|
| Sense Sequence: | |
| 5′ CAACGCACCGAAUAGUUACtt 3′ | (SEQ ID NO:7) |
| Antisense Sequence: | |
| 5′ GUAACUAUUCGGUGCGUUGtt 3′ | (SEQ ID NO:8) |

| | |
|---|---|
| Sense Sequence: | |
| 5′ CGGAAGGUCCCUCAGACAUtt 3′ | (SEQ ID NO:9) |
| Antisense Sequence: | |
| 5′ AUGUCUGAGGGACCUUCCGtt 3′ | (SEQ ID NO:10) |

| | |
|---|---|
| Sense Sequence: | |
| 5′ CAACGCACCUAAUAGUUACtt 3′ | (SEQ ID NO:11) |
| Antisense Sequence: | |
| 5′ GUAACUAUUAGGUGCGUUGtt 3′ | (SEQ ID NO:12) |

| | |
|---|---|
| Sense sequence: | |
| 5′ PGGCUACGUCCAGGAGCGCACC 3′ | (SEQ ID NO:13) |
| Antisense sequence: | |
| 5′ PUGCGCUCCUGGACGUAGCCUU 3′ | (SEQ ID NO:14) |

| | |
|---|---|
| Sense sequence: | |
| 5′ GCUGCACCUGACGCCCUUCtt 3′ | (SEQ ID NO:15) |
| Antisense sequence: | |
| 5′ GAAAGGGCGUCAGGUGCAGCtt 3′ | (SEQ ID NO:16) |

1. A method of detecting adriamycin resistance in a test neoplastic cell from a non-hematological cancer, comprising:
a) measuring a level of p16 expression in the test neoplastic cell of a given origin or cell type; and
b) comparing the level of p16 expression present in the test neoplastic cell to the level of p16 expression in a nonresistant neoplastic cell of the same origin or cell type,
wherein the test neoplastic cell is adriamycin-resistant if the level of p16 expression is greater than the level of p16 expression in the nonresistant neoplastic cell of the same origin or cell type.

2. The method of claim 1, wherein the non-hematological cancer is a solid tumor.

3. The method of claim 2, wherein the solid tumor is a cancer of a tissue selected from the group consisting of breast, ovary, prostate, brain and lung.

4. The method of claim 1, wherein the level of p16 protein is measured.

5. The method of claim 4, wherein the level of p16 protein is measured using a SITp16 test.

6. The method of claim 1, wherein the level of p16 mRNA is measured.

7. The method of claim 6, wherein the level of p16 mRNA is measured using a p16 mRL test.

8. A method of treating or alleviating the symptoms of an adriamycin-resistant non-hematological cancer in a patient comprising, administering to the patient a therapeutically effective amount of an agent that inhibits p16.

9. The method of claim 8, further comprising administering a therapeutically effective amount of adriamycin.

10. The method of claim 8, wherein the agent is selected from the group consisting of a p16 siRNA, a p16-specific antibody, and a p16 antisense nucleic acid molecule.

11. The method of claim 8, wherein the non-hematological cancer is a solid tumor.

12. The method of claim 11, wherein the solid tumor is a cancer of a tissue selected from the group consisting of breast, ovary, prostate, brain and lung.

13. A therapeutic composition, comprising:
a) an agent that inhibits p16; and
b) a pharmaceutically acceptable carrier.

14. The therapeutic composition of claim 13, further comprising adriamycin.

15. The therapeutic composition of claim 13, wherein the agent is selected from the group consisting of a p16 siRNA, a p16-specific antibody, and a p16 antisense nucleic acid molecule.

16. A method for determining if a cancer in a cancer patient is treatable with adriamycin, comprising:
a) measuring a level of p16 expression in a test neoplastic cell from the patient; and
b) comparing the level of p16 expression present in the test neoplastic cell to the level of p16 expression in a nonresistant neoplastic cell of the same origin or cell type,
wherein the cancer is not treatable if the level of p16 expression in the test neoplastic cell is greater than the level of p16 expression in the nonresistant neoplastic cell of the same origin or cell type.

17. The method of claim 16, wherein the non-hematological cancer is a solid tumor.

18. The method of claim 17, wherein the solid tumor is a cancer of a tissue selected from the group consisting of breast, ovary, prostate, brain and lung.

19. The method of claim 16, wherein the level of p16 protein is measured.

20. The method of claim 19, wherein the level of p16 protein is measured using a SITp16 test.

21. The method of claim 16, wherein the level of p16 mRNA is measured.

22. The method of claim 21, wherein the level of p16 mRNA is measured using a p16 mRL test.

23. A method of treating a non-hematological cancer in a cancer patient so as to increase the likelihood of efficacy of a chemotherapeutic agent comprising:
a) detecting adriamycin resistance in a cancer cell from the cancer patient, wherein adriamycin resistance is detected when the level of p16 expression in the the cancer cell from the cancer patient is greater than the level of p16 expression in a nonresistant cancer cell of the same tissue or cell type; and
b) administering to the cancer patient a therapeutically effective amount of an agent that inhibits p16.

24. The method of claim 23, further comprising administering a therapeutically effective amount of adriamycin.

25. The method of claim 23, wherein the agent is selected from the group consisting of a p16 siRNA, a p16-specific antibody, and a p16 antisense nucleic acid molecule.

26. The method of claim 23, wherein the non-hematological cancer is a solid tumor.

27. The method of claim 26, wherein the solid tumor is a cancer of a tissue selected from the group consisting of breast, ovary, prostate, brain and lung.
